SEQUENCE LISTING (34 sequences)

SEQ ID NO: 1 - DNA, 22 bp, synthetic construct
  misc_feature 1..22: note = Target sense sequence of shBCL11B#1
  source 1..22: mol_type = other DNA
tgagccttccagctacatttgc

SEQ ID NO: 2 - DNA, 30 bp, synthetic construct
  misc_feature 1..30: note = Target sense sequence of shBCL11B#2
  source 1..30: mol_type = other DNA
gttctgcggcaagaccttcaagttccagag

SEQ ID NO: 3 - DNA, 30 bp, synthetic construct
  misc_feature 1..30: note = Target sense sequence of shBCL11B#3
  source 1..30: mol_type = other DNA
gaccttcaagttccagagcaatctcatcgt

SEQ ID NO: 4 - DNA, 21 bp, synthetic construct
  misc_feature 1..21: note = Target sense sequence of shBCL11B#4
  source 1..21: mol_type = other DNA
gttcaagaactgcagcaactt

SEQ ID NO: 5 - DNA, 30 bp, synthetic construct
  misc_feature 1..30: note = Target sense sequence of shBCL11B#5
  source 1..30: mol_type = other DNA
cagatcggcaaggaggtgtaccgctgcgac

SEQ ID NO: 6 - DNA, 29 bp, synthetic construct
  misc_feature 1..29: note = Target sense sequence of shBCL11B#6
  source 1..29: mol_type = other DNA
cctaacctgtgtctgcgaagtcctatgga

SEQ ID NO: 7 - DNA, 21 bp, synthetic construct
  misc_feature 1..21: note = Target sense sequence of shBCL11B#7
  source 1..21: mol_type = other DNA
tggaaacccgagggttgatta

SEQ ID NO: 8 - DNA, 20 bp, synthetic construct
  misc_feature 1..20: note = Target sense sequence of shBCL11B-A
  source 1..20: mol_type = other DNA
  misc_feature 17: note = t is urasil.
  misc_feature 19: note = t is urasil.
cagatcggcaaggaggtgta

SEQ ID NO: 9 - DNA, 20 bp, synthetic construct
  misc_feature 1..20: note = Target sense sequence of shBCL11B-B
  source 1..20: mol_type = other DNA
  misc_feature 1..20: note = t is urasil.
cctaacctgtgtctgcgaag

SEQ ID NO: 10 - DNA, 19 bp, synthetic construct
  misc_feature 1..19: note = Target sense sequence of shBCL11B-C
  source 1..19: mol_type = other DNA
gaaactagcggtgttcttt

SEQ ID NO: 11 - DNA, 19 bp, synthetic construct
  misc_feature 1..19: note = Target sense sequence of shBCL11B-D
  source 1..19: mol_type = other DNA
ctgagattgagtgtcagta

SEQ ID NO: 12 - RNA, 23 nt, synthetic construct
  modified_base 6: mod_base = OTHER; note = thymine
  modified_base 8: mod_base = OTHER; note = thymine
  misc_feature 1..23: note = sgRNA#1
  source 1..23: mol_type = other RNA
ggcaatgtcccgccgcaaacagg

SEQ ID NO: 13 - RNA, 23 nt, synthetic construct
  modified_base 6..7: mod_base = OTHER; note = thymine
  modified_base 12: mod_base = OTHER; note = thymine
  modified_base 14..16: mod_base = OTHER; note = thymine
  misc_feature 1..23: note = sgRNA#2
  source 1..23: mol_type = other RNA
gcgggttgccctgtttgcggcgg

SEQ ID NO: 14 - DNA, 63 bp, synthetic construct
  misc_feature 1..63: note = CD8 leader
  source 1..63: mol_type = other DNA
atggccttaccagtgaccgccttgctcctgccgctggccttgctgctccacgccgccaggccg

SEQ ID NO: 15 - DNA, 789 bp, synthetic construct
  misc_feature 1..789: note = CD19 scFv
  source 1..789: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 16 - DNA, 723 bp, synthetic construct
  misc_feature 1..723: note = MSLN scFv
  source 1..723: mol_type = other DNA
caagtccaactcgttcaatcaggcgcagaagtcgaaaagcccggagcatcagtcaaagtctcttgcaaggcttccggctacaccttcacggactactacatgcactgggtgcgccaggctccaggccagggactggagtggatgggatggatcaacccgaattccgggggaactaactacgcccagaagtttcagggccgggtgactatgactcgcgatacctcgatctcgactgcgtacatggagctcagccgcctccggtcggacgataccgccgtgtactattgtgcgtcgggatgggacttcgactactgggggcagggcactctggtcactgtgtcaagcggaggaggtggatcaggtggaggtggaagcgggggaggaggttccggcggcggaggatcagatatcgtgatgacgcaatcgccttcctcgttgtccgcatccgtgggagacagggtgaccattacttgcagagcgtcccagtccattcggtactacctgtcgtggtaccagcagaagccggggaaagccccaaaactgcttatctatactgcctcgatcctccaaaacggcgtgccatcaagattcagcggttcgggcagcgggaccgactttaccctgactatcagcagcctgcagccggaagatttcgccacgtactactgcctgcaaacctacaccaccccggacttcggacctggaaccaaggtggagatcaag

SEQ ID NO: 17 - DNA, 723 bp, synthetic construct
  misc_feature 1..723: note = HER2 scFv
  source 1..723: mol_type = other DNA
caggtgcagctgcagcagagcggccctgagctgaagaagcccggcgagacagtcaagatcagctgcaaggccagcggctaccccttcaccaactacggcatgaactgggtgaaacaggccccaggccagggactgaagtggatgggctggatcaacaccagcaccggcgagagcaccttcgccgacgacttcaagggcagattcgacttcagcctggaaaccagcgccaacaccgcctacctgcagatcaacaacctgaagagcgaggacagcgccacctacttttgcgccagatgggaggtgtaccacggctacgtgccctactggggccagggcaccaccgtgaccgtgtccagcggcggagggggctctggcggcggaggatctgggggagggggcagcgacatccagctgacccagagccacaagtttctgagcaccagcgtgggcgaccgggtgtccatcacctgcaaagccagccaggacgtgtacaacgccgtggcctggtatcagcagaagcctggccagagccccaagctgctgatctacagcgccagcagccggtacaccggcgtgcccagcaggttcaccggcagcggcagcggcccagacttcaccttcaccatcagcagcgtgcaggccgaggacctggccgtgtacttctgccagcagcacttccggacccccttcaccttcggctccggcaccaagctggaaatcaag

SEQ ID NO: 18 - DNA, 135 bp, synthetic construct
  misc_feature 1..135: note = CD8 hinge
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 19 - DNA, 72 bp, synthetic construct
  misc_feature 1..72: note = CD8 Transmembrane domain
  source 1..72: mol_type = other DNA
atctacatctgggcgcccttggccgggacttgtggggtccttctcctgtcactggttatcaccctttactgc

SEQ ID NO: 20 - DNA, 141 bp, synthetic construct
  misc_feature 1..141: note = Fc gamma receptor
  source 1..141: mol_type = other DNA
agacgactcaagatccaggtccgaaaggcagctatagccagccgtgagaaagcagatgctgtctacacgggcctgaacacccggagccaggagacatatgagactctgaagcatgagaaaccaccccagggatccggaagt

SEQ ID NO: 21 - DNA, 123 bp, synthetic construct
  misc_feature 1..123: note = CD28 Intracellular domain
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 22 - DNA, 336 bp, synthetic construct
  misc_feature 1..336: note = CD3 zetta
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 23 - DNA, 575 bp, synthetic construct
  misc_feature 1..575: note = IRES
  source 1..575: mol_type = other DNA
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 24 - DNA, 717 bp, synthetic construct
  misc_feature 1..717: note = GFP
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 25 - DNA, 600 bp, synthetic construct
  misc_feature 1..600: note = RHA
  source 1..600: mol_type = other DNA
aaaggcggctgcggaatcctaggactggcgcggccggcaccctgctgggccgggcgcagggaccggggacccggagccggcggccgcccctggccctacggctcccccagccggaacgcaccccgctctcctccgcccgcgccgtcagcgcggacccacgcgctcgccaacttttccccacttcccggctccccctccccctccgctcccccagccccctccccctcctctccaaaccccgccaccagcgccgccgccgccacacacgccgctcggaggggcgagcgtccagccgggctcggcgcgcacacacacacactcctccagcctgcatgccccctccccggcccggagccggctccgcaggcccccgagcccgaggcgcgtccggctgctcggcgccccaactccccgggctgcaaagaaactttcctatggcccccgccccccgccatgctccaggccgacgccgtagactctgccagccagcgggcggccccggcgcctggccaggctcggctgttccgggctcggtgtccccagccccagacgcccggagccccatctccggcccctcgcgcgcactccgcagacacttac

SEQ ID NO: 26 - DNA, 600 bp, synthetic construct
  misc_feature 1..600: note = LHA
  source 1..600: mol_type = other DNA
tgccccggcatctattctggcatcgcccggagagctgcactgatggggggagccgggggagggggtccgagccgccgccgcgccgctgccgccgctgccgccgccgccgccgccgccgcacctcctcctctgcccgggttggtgttttttttcccttcctctctttccctctcttcctcctcttcttcttctttattttgctctttcttctatgctgttttttgttttgtttgcaaaaagaaaaaaaagggaagaaaagcaagaaaaacctctcgatctaaaataagaaaaagaggcaaaaaaaaaaaaaactgctgttgctttccgcggactggctggtttctttaaaaatatattctttcgaaggaaaaaaaatctcttacacttcttcaaactgcttggcctcttgcacttgcaaatgtcttcttgaacttaaactgggttttgcaccggctcctgacactttctttcagggtctggtaggtggaaagcgcacttctaccaggaggggaaaaaaaatgcaaacaaataaaaaaataaaagaagaaaaagcaaaggaaaaaaaaaagcaaagaaaacttggggacttgtctcgtaccc

SEQ ID NO: 27 - DNA, 408 bp, synthetic construct
  misc_feature 1..408: note = SFFV promoter
  source 1..408: mol_type = other DNA
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 28 - DNA, 575 bp, synthetic construct; identical to SEQ ID NO: 23
  misc_feature 1..575: note = Poly A signal
  source 1..575: mol_type = other DNA

SEQ ID NO: 29 - DNA, 20 bp, synthetic construct
  misc_feature 1..20: note = LHA primer
  source 1..20: mol_type = other DNA
accgaaccggggcagtttta

SEQ ID NO: 30 - DNA, 22 bp, synthetic construct
  misc_feature 1..22: note = SFFV promoter primer
  source 1..22: mol_type = other DNA
ttttcatgtacccgcccttgat

SEQ ID NO: 31 - DNA, 20 bp, synthetic construct
  misc_feature 1..20: note = LMP-1 Forward
  source 1..20: mol_type = other DNA
tcctcctgtttctggcgatt

SEQ ID NO: 32 - DNA, 21 bp, synthetic construct
  misc_feature 1..21: note = LMP-1 Reverse
  source 1..21: mol_type = other DNA
ggagtcatcgtggtggtgttc

SEQ ID NO: 33 - DNA, 21 bp, synthetic construct
  misc_feature 1..21: note = GFP Forward
  source 1..21: mol_type = other DNA
atggtgagcaagggcgaggag

SEQ ID NO: 34 - DNA, 23 bp, synthetic construct
  misc_feature 1..23: note = GFP Reverse
  source 1..23: mol_type = other DNA
cggtggtgcagatgaacttcagg

(19) United States
(12) Patent Application Publication
(10) Pub. No.: US 2024/0318139 A1
(43) Pub. Date: Sep. 26, 2024

(54) METHOD FOR PRODUCING DIRECTLY REPROGRAMMED NATURAL KILLER CELLS AND USES THEREOF

(71) Applicant: KOREA RESEARCH INSTITUTE OF BIOSCIENCE AND BIOTECHNOLOGY, Daejeon (KR)

(72) Inventors: Yee Sook CHO, Daejeon (KR); Han-Seop KIM, Daejeon (KR); Jae Yun KIM, Daejeon (KR); Binna SEOL, Daejeon (KR)

(21) Appl. No.: 18/599,724

(22) Filed: Mar. 8, 2024

Related U.S. Application Data
(63) Continuation-in-part of application No. PCT/KR2022/013561, filed on Sep. 8, 2022.

(30) Foreign Application Priority Data
Sep. 10, 2021 (KR) 10-2021-0121118

(51) Int. Cl. C12N 5/0783; A61K 39/00; A61P 35/00; C07K 14/705; C07K 14/725; C07K 16/30; C12N 9/22; C12N 15/11; C12N 15/113; C12N 15/90

(52) U.S. Cl. CPC C12N 5/0646; A61K 39/4613; A61K 39/4631; A61K 39/464468; A61P 35/00; C07K 14/7051; C07K 14/70517; C07K 16/30; C12N 9/22; C12N 15/11; C12N 15/113; C12N 15/907; C07K 2317/53; C07K 2317/622; C07K 2317/73; C07K 2319/03; C12N 2310/14; C12N 2310/20; C12N 2310/531; C12N 2501/125; C12N 2501/2302; C12N 2501/2307; C12N 2501/2315; C12N 2501/727; C12N 2510/00

(57) ABSTRACT

The present invention relates to a method for preparing directly reprogrammed natural killer (drNK) cells or CAR (chimeric antigen receptor) gene introduced CAR-drNK cells using substances and methods that inhibit BCL11B (B-cell leukemia 11B) gene expression and/or function. The present invention also relates to drNK cells or CAR-drNK cells prepared by a BCL11B gene-based cell reprogramming method, and a cell therapeutic and/or a composition for the prevention or treatment of cancer diseases and infectious diseases caused by viruses, bacteria, fungi, and the like, and/or inflammatory diseases, which contain the cells.

### CROSS-REFERENCE TO RELATED APPLICATIONS

This application is a continuation-in-part of International Appl. PCT/KR2022/013561, filed Sep. 8, 2022, which claims priority from Korean Appl. No. 10-2021-0121118, filed Sep. 10, 2021, all of which are hereby incorporated by reference.

### TECHNICAL FIELD

The present invention relates to a method for preparing directly reprogrammed natural killer (drNK) cells or CAR (chimeric antigen receptor) gene-introduced CAR-drNK cells using substances and methods that inhibit BCL11B (B-cell leukemia 11B) gene expression and/or function. The present invention also relates to drNK cells or CAR-drNK cells prepared by a cell reprogramming method, and a cell therapeutic and/or a composition for the prevention or treatment of cancer diseases, infectious diseases caused by viruses, bacteria, fungi, and the like, and/or inflammatory diseases, which contain the cells.

### BACKGROUND ART

NK (natural killer) cells are a type of lymphocyte blood cell that plays an important role in both innate and adaptive immune responses, and particularly have the function of recognizing and immediately eliminating abnormal cells, such as cancer cells and cells infected with viruses, bacteria, fungi, parasites, and the like. Additionally, studies to develop therapeutics using this function are actively underway.

As it has been revealed that NK cells not only suppress the development, proliferation, and metastasis of cancer cells, but can also effectively control cancer stem cells and thus have a function of preventing cancer recurrence, the importance of studies to develop anticancer drugs using NK cells is growing. Recently, in terms of functionality, as an innovative method to enhance the efficacy of anticancer treatment, CAR-NK platform technology, which introduces cancer cell-targeting chimeric antigen receptor (CAR) genes that are effective in promoting specificity and activation against target cancer cells, is being actively developed. Unlike CAR-T cells, another immune cell therapeutic, as CAR-NK cells have been reported to have characteristics of being less likely to cause cytokine release syndrome (CRS) [K. Rezvani, et al., Mol. Ther., 25 (2017), pp. 1769-1781], rarely causing immunosuppression, and improving anti-PD-1 immunotherapy. The advantages of developing therapeutics that can overcome the issues of existing cell therapeutics and maximize clinical benefits are being highlighted [K. C. Barry, et al., Nat. Med., 24 (2018), pp. 1178-1191]. In preclinical and clinical trials, it has been found that CAR-NK can effectively eliminate not only blood cancer but also hard-to-treat solid cancer cells [E. L. Siegler, et al., Cell Stem Cell, 23 (2018), pp. 160-161], and CAR-NK's potential to be developed as an effective anticancer immune cell therapeutic with improved functionality and fewer side effects for a wide range of cancer diseases is recognized.

Currently, immortalized NK-92 cells and pluripotent stem cells [PSC: embryonic stem cell (ESC) and induced pluripotent stem cell (iPSC)], which have excellent proliferative ability, are used as the main cell source for CAR-NK construction. NK-92 cells are an immortalized cell line, so continuous production thereof is easy, but NK-92 cells are inherently cancer cells derived from patients with non-Hodgkin's lymphoma so there are safety issues and the disadvantage of a low anticancer effect in the body has been pointed out. For CAR-NK production based on pluripotent stem cells, CAR-PSCs loaded with the CAR gene are first required to be produced, undergo isolation and amplification, and then additionally undergo a step-by-step differentiation process into CAR-NK cells. NK cell production using PSC has the advantage of being able to mass proliferate and bank initial cells, but the problem has been pointed out that the process for producing CAR-NK, the final product, from PSC is complicated, takes a lot of time, and costs a lot of money. In addition to this, residual undifferentiated PSCs have the potential to form tumors, and it is essential to previously secure the technology for securing and maintaining safety and control the quality in order to use PSC-derived differentiated cells for treatment purposes.

Recently, along with the development of somatic cell reprogramming technology, the development of technology to directly produce functional cells having high clinical utility without going through the production process of stem cells such as iPSC is actively underway. Functional cells produced through direct reprogramming technology have been highlighted for their technological advantages in that the cells have a low risk of epigenetic remodeling and tumor formation, and it is easy to enhance safety, reliability, and efficiency by simplifying the cell production process. These characteristics are expected to ultimately contribute to eliminating barriers to commercialization by dramatically shortening and reducing the time and cost required to develop therapeutics, and research and development efforts are continuously increasing to secure raw materials for cell therapeutics targeting various diseases, including cancer diseases, by the use of direct reprogramming technology.

To date, there has been no report on the effect of NK cells produced through direct reprogramming on the prevention, treatment, or improvement of infections caused by viruses, bacteria, fungi, and the like, and/or inflammation.

Against this background, the present inventors have made diligent efforts to solve various problems in the production of NK-based therapeutics by developing a new approach to secure NK cells or CAR-NK cells through direct reprogramming without going through the iPSC reprogramming process and PSC differentiation process unlike the existing method to secure NK cells through a PSC reprogramming process.

### DISCLOSURE

### Technical Problem

The present inventors have discovered that drNK cells or CAR-drNK cells can be produced from isolated human somatic cells through somatic cell reprogramming culture by controlling the expression of the BCL11B (B-cell leukemia 11B) gene. The present inventors have also discovered that cells produced by the method exhibit cancer cell-killing ability and antiviral, antibacterial, and antifungal effects and can be applied to the prevention or treatment of cancer, infectious diseases, and/or inflammatory diseases, and thereby completing the present invention.

### Technical Solution

An object of the present invention is to provide a method for preparing a directly reprogrammed natural killer (drNK) cell, the method including a) inhibiting BCL11B gene expression in an isolated cell; and b) culturing the cell in step a) in a medium containing a cytokine and a growth factor to convert the cell into an NK (natural killer) cell.

Another object of the present invention is to provide a drNK cell prepared by the method.

Still another object of the present invention is to provide a method for preparing a CAR-drNK cell, the method including additionally introducing a CAR gene in any one or more steps selected from a) or b) in the method.

Still another object of the present invention is to provide a CAR-drNK cell prepared by the method.

Still another object of the present invention is to provide a cell therapeutic composition for prevention or treatment of cancer, containing a cell prepared by the method as an active ingredient.

Still another object of the present invention is to provide a pharmaceutical composition for prevention or treatment of cancer, containing a cell prepared by the method as an active ingredient.

Still another object of the present invention is to provide a cell therapeutic composition for prevention or treatment of an infectious disease and/or an inflammatory disease, containing a cell prepared by the method as an active ingredient.

Still another object of the present invention is to provide a pharmaceutical composition for prevention or treatment of an infectious disease and/or an inflammatory disease, containing a cell prepared by the method as an active ingredient.

Still another object of the present invention is to provide a method for treating cancer, the method including administering the cell therapeutic composition or pharmaceutical composition to a subject.

Still another object of the present invention is to provide a method for treating an infectious disease and/or an inflammatory disease, the method including administering the cell therapeutic composition or pharmaceutical composition to a subject.

### Advantageous Effects

The drNK cells or CAR-drNK cells constructed through the present invention have excellent cell-killing ability against cancer cells or cells infected with viruses, bacteria, and fungi, and can therefore be applied as a cell therapeutic and a composition for the prevention or treatment of cancer, or infectious diseases caused by bacteria and fungi, and/or inflammatory diseases.

### BRIEF DESCRIPTION OF DRAWINGS

FIG. 1A is a schematic diagram illustrating a method for preparing shBCL11B-drNK cells and FIG. 1B is a schematic diagram illustrating a method for preparing CAR-shBCL11B-drNK cells;

FIG. 2A is a schematic diagram illustrating a method for preparing gBCL11B-drNK cells using genetic scissors CRISPR/Cas9, and FIG. 2B is a schematic diagram illustrating a method for preparing CAR-gBCL11B-drNK cells using genetic scissors CRISPR/Cas9;

FIG. 3A is a schematic diagram illustrating a lentiviral vector encoding CAR and FIG. 3B is a schematic diagram illustrating an AAV vector containing a genetic scissors plasmid vector targeting the exon1 region of the BCL11B gene and an insertion gene expressing CAR;

FIG. 4A illustrates the sequences and positions of seven types of shRNAs (shBCL11B#1, shBCL11B#2, shBCL11B#3, shBCL11B#4 shBCL11B#5, shBCL11B#6, and shBCL11B#7) and four types of siRNAs (siBCL11B-A, siBCL11B-B, siBCL11B-C, and siBCL11B-D), FIG. 4B is a schematic diagram illustrating the preparation of shBCL11B/siBCL11B-drNK cells from PBMC, and FIG. 4C illustrates the results of examining NK cell production using an NK marker;

FIGS. 5A and 5B are diagrams illustrating the effect of culture components of the first medium and second medium on the production yield of shBCL11B-drNK cells;

FIG. 6 illustrates the results of verifying the expression characteristics of NK-specific markers in shBCL11B-drNK cells;

FIG. 7 illustrates the results of analyzing the cancer cell-killing ability of shBCL11B-drNK cells;

FIG. 8 illustrates the results of comparing the cancer cell-killing ability between shBCL11B-drNK cells and NK-92 cells;

FIGS. 9A and 9B illustrate the results of CD107a⁺ cell frequency and IFN-gamma expressing cell frequency for cancer cell sensitization of shBCL11B-drNK cells and PBMC-NK cells;

FIGS. 10A and 10B illustrate the results of comparing the in vivo cancer cell-killing ability between shBCL11B-drNK cells and PBMC-NK cells in a mouse prostate cancer model (PC-3) and a mouse ovarian cancer model (SK-OV-3);

FIG. 11A is a schematic diagram illustrating a method for preparing CAR-shBCL11B-drNK cells from PBMC and FIG. 11B illustrates the results of examining the production of CAR-shBCL11B-drNK cells using an NK marker;

FIG. 12 illustrates the results of verifying the expression characteristics of NK-specific markers in CAR (MSLN-CAR)-shBCL11B-drNK cells;

FIG. 13A is a schematic diagram illustrating the preparation of gBCL11B-drNK cells from PBMC and FIG. 13B illustrates the results of examining NK cell production using an NK marker;

FIG. 14 illustrates the results of verifying the expression characteristics of NK-specific markers in gBCL11B-drNK cells;

FIGS. 15A and 15B are schematic diagrams illustrating a method for preparing CAR-gBCL11B-drNK cells from PBMC, FIG. 15C illustrates the results of examining NK cell production using an NK marker, and FIG. 15D illustrates the results of examining CAR-KI (knock-in) inserted into the genome;

FIG. 16A illustrates the cancer cell-killing ability of shBCL11B-drNK, gBCL11B-drNK, CAR(MSLN-CAR)-shBCL11B-drNK, and CAR(MSLN-CAR)-gBCL11B-drNK cells and FIG. 16B illustrates the results of CD107a⁺ cell frequency;

FIG. 17 illustrates the results of analyzing the antiviral efficacy of shBCL11B-drNK cells, in which FIG. 17A illustrates the cell-killing ability against B-lymphoma Raji cells infected with Epstein-Barr virus (EBV), FIG. 17B illustrates the CD107a⁺ cell frequency in EBV-infected Raji cells, FIG. 17C illustrates the expression level of LMP-1 (latent membrane protein 1) in EBV-infected Raji cells upon co-culture with shBCL11B-drNK, NK-92, and PBMC-NK cells, and FIG. 17D illustrates the results of analyzing the antiviral efficacy against cells infected with human immunodeficiency virus (HIV), influenza virus, papilloma virus, and hepatitis virus;

FIG. 18 illustrates the results of analyzing apoptosis obtained by co-culture of shBCL11B-drNK cells and cells infected with the SARS-CoV-2 virus;

FIG. 19 illustrates the results of analyzing the antibacterial efficacy against Gram-negative bacteria and Gram-positive bacteria obtained by shBCL11B-drNK cell culture, in which FIGS. 19A and 19B illustrate the number of colonies of Escherichia coli, a Gram-negative bacterium, and the CD107a⁺ cell frequency obtained by shBCL11B-drNK cell culture and FIG. 19C illustrates the results of the CD107a⁺ cell frequency of streptococci, a Gram-positive bacterium, obtained by shBCL11B-drNK cell culture;

FIG. 20 illustrates the results of the CD107a⁺ cell frequency obtained by co-culture of shBCL11B-drNK cells and Candida albicans;

FIG. 21 illustrates the results of analyzing the antifungal activity of shBCL11B-drNK against Aspergillus fumigatus;

FIG. 22 is a diagram illustrating a lentiviral vector expressing MSLN-CAR;

FIG. 23 is a diagram illustrating a CAR plasmid expressing MSLN-CAR; and

FIG. 24 is a diagram illustrating an AAV plasmid expressing MSLN-CAR.

### BEST MODE FOR CARRYING OUT THE INVENTION

The present invention will be specifically described as follows. Meanwhile, each description and embodiment disclosed in the present disclosure may also be applied to other descriptions and embodiments. That is, all combinations of various elements disclosed in the present disclosure fall within the scope of the present disclosure. Further, the scope of the present disclosure is not limited by the specific description below.

An aspect of the present invention provides a method for preparing a directly reprogrammed natural killer (drNK) cell, the method including a) inhibiting BCL11B gene expression in an isolated cell; and b) culturing the cell in step a) in a medium containing a cytokine and a growth factor to convert the cell into an NK (natural killer) cell.

A specific embodiment of the present invention may be a method for preparing a directly reprogrammed natural killer (drNK) cell, which includes a) inhibiting BCL11B gene expression in an isolated cell by introducing any one or more selected from the following i) to iii) into the cell: i) shRNA (short hairpin RNA), ii) siRNA (short interfering RNA), and iii) CRISPR (clustered regularly interspaced short palindromic repeats)/Cas system; and b) culturing the cell in step a) in a medium containing a cytokine and a growth factor to convert the cell into an NK (natural killer) cell, but is not limited thereto.

In the present invention, the term “NK (natural killer) cell” refers to a key innate immune cell that immediately recognizes and eliminates infections by viruses, bacteria, fungi, and parasites, as well as abnormal autologous cells (particularly cancer cells). Unlike T cells, which recognize target cells by expressing antigen-specific receptors, NK cells recognize abnormal changes in target cells (particularly cancer cells and infected cells), including the balance of inhibitory or activating receptors, such as killer immunoglobulin receptors (KIR), natural cytotoxicity receptors (NCR), DNAX accessory molecule-1 (DNAM-1), and NK group 2 member D (NKG2D), and the loss of surface MHC (major histocompatibility complex) class I antigen without antigen specificity and human leukocyte antigen (HLA) matching, and exhibit contact-dependent cytotoxicity through various mechanisms. Unlike T cells, which may cause graft-versus-host disease (GVHD) against non-self allogeneic cells that do not match human leukocyte antigens (HLA), it has been found that allogeneic NK cells have almost no side effects such as graft-versus-host disease and have a strong anticancer effect.

Recently, as a way to promote the specificity and activation of NK cells towards disease target cells, studies are being actively conducted to construct NK (CAR-NK) cells that express a chimeric antigen receptor (CAR) specific to the target cell antigen and investigate the enhancement of target cell-killing activity and therapeutic efficacy of the NK cells. As a method for preparing the CAR-NK cells, a method in which the CAR gene is introduced into a single NK cell line (NK-92, embryonic stem cell, induced pluripotent stem cell, or the like) that is relatively easily proliferated is mainly used, but low production efficiency, a series of complex processes, safety issues (possibility of tumor formation, and the like), low treatment efficacy, and the like are still emerging as problems that are required to be overcome.

In the present invention, the term “direct reprogramming” refers to a method in which the global gene expression pattern and the like of a particular cell are regulated to convert the lineage into a desired cell having completely different characteristics. The direct reprogramming may be a concept including reprogramming, differentiation, direct differentiation, dedifferentiation, direct dedifferentiation, conversion, direct conversion, trans-differentiation, or direct trans-differentiation of a cell, but is not limited thereto.

The “direct reprogramming” may involve performing “cell-fate conversion” by introducing an oligonucleotide or vector containing a foreign gene or DNA into a cell, and may mean changing the state of a cell to a different state. The “differentiation” refers to a phenomenon in which daughter cells created by cell division acquire functions different from the original functions of the parent cell. In the present invention, the term “direct reprogramming” can be used interchangeably with “direct cell-fate conversion induction”, “direct cell-fate conversion”, and “cell-fate conversion”.

In the present invention, the term “differentiated cells” refers to cells with specialized structures or functions, that is, a state in which biological cells, tissues, and the like are changed into appropriate forms and functions to perform the roles assigned thereto. For example, “differentiated cells” may be broadly classified as differentiated ectoderm, mesoderm, and endoderm cells derived from pluripotent stem cells such as embryonic stem cells, and narrowly as differentiated red blood cells, white blood cells, platelets, and the like derived from hematopoietic stem cells.

In the present invention, the term “lineage-converted cells” refers to cells converted into a cell type with different lineage characteristics as the inherent lineage characteristics thereof are changed embryologically or artificially (for example, direct cell-fate conversion induction and reprogramming), and they have characteristics of a cell type that is completely different from the cell type before conversion. In the present invention, the lineage-converted cells may be target cells. For example, peripheral blood mononuclear cells may be converted into lymphoid stem cells, specifically NK cells, which are a different lineage from peripheral blood mononuclear cells, through direct cell-fate conversion induction, but the lineage-converted cells are not limited thereto.

As described above, NK cells are produced through primary isolation and culture of human-derived NK cells, differentiation from stem cells, somatic cell reprogramming, or the like for use as immune cell therapeutics, and the like. In particular, in order to prepare NK cells using induced pluripotent stem cell (iPSC) reprogramming technology, it is required to go through 1) first, a process of preparing induced pluripotent stem cells from isolated somatic cells, 2) a process of differentiating hematopoietic stem (progenitor) cells, which are differentiation intermediates, from the induced pluripotent stem cells, and 3) additionally, a process of inducing NK cell differentiation. As such, iPSC-NK production technology based on conventional reprogramming technology is required to sequentially go through complex culture and differentiation processes, and thus has disadvantages of low preparation efficiency, long time, and high cost. Since the iPSC-NK is prepared via induced pluripotent stem cells with pluripotency, the remaining undifferentiated cells are closely related to the possibility of tumor formation, and securing safety has emerged as an important issue that is required to be verified.

In contrast, in the present invention, NK cells are prepared directly from isolated somatic cells through direct reprogramming induction without going through iPSCs, thus they have advantages of short preparation time and reduced cost as well as secured safety, are different from NK cells of the prior art, and can provide an alternative that can overcome the problems of existing NK cell sources.

Specifically, differing from existing cell reprogramming methods utilizing overexpression of multiple pluripotency transcription factor gene combinations, in the present invention, “directly reprogrammed natural killer (drNK)” cells have been constructed using substances and methods that inhibit the single BCL11B (B-cell leukemia 11B) gene expression and/or function or “CAR-drNK” cells that express CAR have been constructed by additionally introducing the CAR gene into the drNK cells, and it has been thus found that NK cells with improved genetic safety can be prepared. In particular, the present invention proposes an innovative alternative that overcomes the random gene insertion problem of existing reprogramming methods by limiting gene sequence editing for BCL11B expression control through incorporation of genetic scissors technology.

In the present invention, the drNK cell preparation method may include the step a) of inhibiting BCL11B gene expression in isolated cells by introducing a direct reprogramming factor into the cells.

The direct reprogramming factor introduced into the isolated cells may be a substance that inhibits BCL11B (B-cell lymphoma/leukemia 11B) gene expression. For example, the direct reprogramming factor may be antisense oligonucleotide, short hairpin RNA (shRNA), short interfering RNA (siRNA), microRNA, or CRISPR/Cas system. More specifically, the direct reprogramming factor may be BCL11B antisense oligonucleotide, BCL11B short hairpin RNA (shRNA), BCL11B short interfering RNA (siRNA), BCL11B microRNA, or CRISPR/Cas9-gRNA-BCL11B, but is not limited thereto.

As an example, the reprogramming factor may be i) BCL11B shRNA (short hairpin RNA), ii) BCL11B siRNA (short interfering RNA), or iii) CRISPR (clustered regularly interspaced short palindromic repeats)/Cas9-gRNA-BCL11B.

In the present invention, the term “isolated cell” is not particularly limited, but may be a cell whose lineage has already been specified, for example a germ cell, somatic cell, or progenitor cell. The “somatic cell” refers to all cells that have completed differentiation and constitute animals and plants, excluding germ cells. The “progenitor cell” refers to a parent cell that does not express a differentiation trait, but has a differentiation fate, in a case where a cell corresponding to the progeny is found to express a specific differentiation trait. For example, hematopoietic stem cells correspond to progenitor cells for blood cells, and mesenchymal stem cells correspond to progenitor cells for mesenchymal cells.

The isolated cells may be, but are not limited to, cells derived from humans, and cells derived from a variety of organisms may also fall within the scope of the present invention. The isolated cells of the present invention may include both in vivo and in vitro cells.

The isolated cells may be somatic cells, as an example, may be somatic cells excluding NK cells as another example, and may be one or more selected from the group consisting of blood cells and fibroblasts as still another example, but are not limited thereto. For example, the blood cells may be peripheral blood mononuclear cells (PBMC), but are not limited thereto.

In the present invention, the term “direct reprogramming inducing factor” refers to a gene (or polynucleotide) that can be introduced into a cell and induce cell-fate conversion, or a protein encoded therefrom. The direct reprogramming inducing factor may vary depending on the target cell to be obtained through reprogramming and the type of cell before cell-fate conversion. For the purpose of the present invention, in order to induce isolated somatic cells into NK cells, the direct reprogramming inducing factor introduced into the isolated somatic cells may be a substance that inhibits BCL11B (B-cell lymphoma/leukemia 11B) gene expression, specifically may be BCL11B antisense oligonucleotide, BCL11B short hairpin RNA (shRNA), BCL11B short interfering RNA (siRNA), BCL11B microRNA, or CRISPR/Cas9-gRNA-BCL11B, but is not limited thereto, and any substance or method known in the art may be included as long as it is a substance or method for inhibiting BCL11B gene expression. Cell-fate conversion using the direct reprogramming inducing factor regulates the overall gene expression pattern of a cell to induce conversion into the target cell. By introducing the direct reprogramming inducing factor into cells and culturing the cells for a certain period of time, the cells can be induced to convert into target cells having the gene expression pattern of the target type of cell. In the present invention, the “direct reprogramming inducing factor” may be used interchangeably with “direct cell-fate conversion inducing factor”, “cell-fate conversion inducing factor”, and “reprogramming factor”.

In the present invention, the term “introduction of a direct reprogramming inducing factor” may refer to a method of increasing or decreasing the expression level of a direct reprogramming inducing factor in a cell through a method in which a direct reprogramming inducing factor is administered to a cell culture solution; a method in which a direct reprogramming inducing factor is injected directly into a cell; a method in which the expression level of a direct reprogramming inducing factor present in a cell is increased or decreased; a method in which an expression vector containing a gene encoding a direct reprogramming inducing factor is transformed into a cell; a method in which a gene sequence is modified to increase or decrease the expression of a gene encoding a direct reprogramming inducing factor; a method in which a foreign expressed gene encoding a direct reprogramming inducing factor is introduced; a method in which treatment is performed with a substance that has the effect of inducing or inhibiting the expression of a direct reprogramming inducing factor; and any combination thereof, but is not limited thereto as long as it can increase or decrease the expression level of a direct reprogramming inducing factor. In particular, the introduction of a direct reprogramming inducing factor may be to induce the expression of the direct reprogramming inducing factor at desired time under desired conditions. Specifically, the method of introducing a direct reprogramming inducing factor into a cell may be a method in which a direct reprogramming inducing factor is administered to a cell culture solution and a method in which an expression vector containing a gene encoding a direct reprogramming inducing factor is transformed into a cell, but is not limited thereto.

As an example, as the method in which a direct reprogramming inducing factor is injected directly into a cell, any method known in the art may be selected and used, and is not limited thereto, but any method can be appropriately selected from among microinjection, electroporation, particle bombardment, direct muscle injection, and methods using an insulator and a transposon and applied.

In the present invention, the direct reprogramming inducing factor that inhibits BCL11B gene expression may be any one or more selected from the following: i) BCL11B shRNA, ii) BCL11B siRNA, or iii) CRISPR/Cas9-gRNA-BCL11B.

In the present invention, the term “shRNA (short hairpin RNA)” is an artificial RNA molecule with a tight hairpin turn that is mainly used to silence target gene expression via RNA interference. For the purpose of the present invention, the shRNA of the present invention may inhibit BCL11B gene expression.

Specifically, the target sense sequences of shRNAs (shBCL11B#1, shBCL11B#2, shBCL11B#3, shBCL11B#4, shBCL11B#5, shBCL11B#6 and shBCL11B#7) of the present invention may each be any one or more selected from the group consisting of SEQ ID NOs: 1 to 7, but are not limited thereto.

In the present invention, the term “siRNA (short interfering RNA)” refers to a nucleic acid molecule capable of mediating RNA interference or gene silencing. For the purpose of the present invention, the siRNA of the present invention may inhibit BCL11B gene expression.

Specifically, the target sense sequences of siRNAs (shBCL11B-A, shBCL11B-B, shBCL11B-C and shBCL11B-D) of the present invention may each be any one or more selected from the group consisting of SEQ ID NOs: 8 to 11, but are not limited thereto.

In the present invention, the term “CRISPR (clustered regularly interspaced short palindromic repeats)/Cas system” is a type of gene editing technology and may be composed of CRISPR protein, Cas protein, and target DNA or RNA. The “CRISPR/Cas system” is a system that can accurately cut target DNA or RNA sequences, and may be a method of performing gene editing by introducing a complex of Cas enzyme and gRNA into cells. Specifically, the Cas protein in the CRISPR/Cas system may be Cas3, Cas9, Cas10, Cas12a, and Cas13. As a specific embodiment, the Cas protein may be Cas9, and in this case, the target RNA may be gRNA (guide RNA), but is not limited thereto.

The CRISPR/Cas9 has the advantage of editing accuracy and a greatly wide applicable range compared to Zinc Finger or TALEN (transcription activator-like effector nuclease). For the purpose of the present invention, CRISPR/Cas9 of the present invention may inhibit BCL11B gene expression, and may include any genetic scissors that inhibit BCL11B gene expression without limitation.

Specifically, the CRISPR/Cas9 system of the present invention may include sgRNA #1 (SEQ ID NO: 12) and sgRNA #2 (SEQ ID NO: 13) targeting Exon1 (BCL11B-ex1) derived from the genomic sequence (NC_000014.9) containing BCL11B, but may include any gRNA targeting a genomic sequence including BCL11B without limitation.

In particular, the present invention is significant in that it overcomes the random gene insertion problem of existing reprogramming methods by incorporating genetic scissors such as the CRISPR/Cas9 system and limiting the BCL11B-specific gene sequence.

In the method of the present invention, the CRISPR/Cas9-gRNA-BCL11B may be introduced in any one or more steps selected from step a) or step b), but is not limited thereto.

The direct reprogramming inducing factor that inhibits BCL11B gene expression of the present invention may be introduced into the target cells (parent cells) for direct reprogramming induction through an antisense oligonucleotide, a plasmid vector or viral vector, or microRNA.

In the present invention, the term “vector” refers to a DNA product containing a suitable regulatory sequence and the base sequence of a target protein or polypeptide so as to express the target protein or polypeptide in a suitable host. The regulatory sequence may contain a promoter, an operator, a start codon, a stop codon, a polyadenylation signal, and an enhancer. The vector of the present invention contains a signal sequence or leader sequence for membrane targeting or secretion in addition to the regulatory sequence and may be constructed in various ways depending on the purpose. The promoter of the vector may be constitutive or inducible. The vector contains a selectable marker for selecting a host cell containing the vector and, if the vector is replicable, an origin of replication. After transformation into a suitable host cell, the vector can replicate or function independently of the host genome and can be integrated into the genome itself.

The vector used in the present invention is not particularly limited as long as it can replicate within the host cell, and any vector known in the art can be used. Examples of commonly used vectors include natural or recombinant viral vectors, episomal vectors, plasmid vectors, cosmid vectors, bacterial artificial chromosomes (BAC), and yeast artificial chromosomes (YAC).

Specifically, the viral vector may include vectors derived from retroviruses such as Sendai virus, lentivirus, HIV (human immunodeficiency virus), MLV (murine leukemia virus), ASLV (avian sarcoma/leukosis), SNV (spleen necrosis virus), RSV (Rous sarcoma virus), and MMTV (mouse mammary tumor virus), adenovirus, adeno-associated virus, herpes simplex virus, and the like, and more specifically, may be an RNA-based viral vector, but is not limited thereto.

The episomal vector is a non-viral, non-insertable vector, and is known to have the property of being able to express the gene contained in the vector without being inserted into the chromosome. Therefore, cells containing the episomal vector may include both a case where the episomal vector is inserted into the genome and a case where the episomal vector exists within the cell without being inserted into the genome.

In the present invention, the term “operably linked” refers to a functional linkage between a nucleic acid expression regulating sequence and a nucleic acid sequence encoding a target protein to perform a general function. The operative linkage with a recombinant vector may be constructed using genetic recombination techniques well known in the art, and enzymes and the like generally known in the art are used for site-specific DNA cutting and pasting.

In the present invention, the method for preparing a drNK cell may include the step b) of culturing the cells in step a) in a medium containing cytokines and growth factors to convert the cells into NK cells.

In the present invention, the term “culture” means growing cells under controlled environmental conditions. The culture process of the present invention may be carried out according to media and culture conditions known in the art. Such a culture process may be easily adjusted by those skilled in the art depending on the cells selected. For the purpose of the present invention, the culture is a process of converting cells into which direct reprogramming or cell-fate conversion inducing factors are introduced into target cells of a different lineage, and the composition of the first medium or second medium for culturing the cells into which a direct reprogramming inducing factor is introduced may be a composition suitable for conversion into target cells. For example, the medium may contain growth factors and cytokines, and may further contain a GSK3β (glycogen synthase kinase 3β) inhibitor, a PDK1 (3-phosphoinositide-dependent kinase 1) inhibitor, and an AHR (aryl hydrocarbon receptor) inhibitor in addition to these, but is not limited thereto.

In the present invention, the medium in b) may contain cytokines and growth factors.

In the present invention, the term “cytokines” refers to a variety of relatively small proteins produced by cells and used in cell signaling, which can affect other cells, including themselves. Cytokines are generally known to be associated with inflammation or immune responses to infection. The cytokines may be, for example, IL (interleukin)-2, IL-3, IL-5, IL-6, IL-7, IL-11, IL-15, IL-21, BMP4 (bone morphogenetic protein 4), activin A, notch ligand, G-CSF (granulocyte-colony stimulating factor), and SDF-1 (stromal cell-derived factor-1), and may specifically be any one or more selected from the group consisting of IL-2, IL-7, IL-15, IL-21, and a combination thereof, but are not limited thereto.

In the present invention, the term “growth factors” refers to polypeptides that promote division, growth and differentiation of various cells. The growth factors may be, for example, EGF (epidermal growth factor), PDGF-AA (platelet-derived growth factor-AA), IGF-1 (insulin-like growth factor 1), TGF-β (transforming growth factor-3), FGF (fibroblast growth factor), SCF (stem cell factor), and FLT3L (FMS-like tyrosine kinase ligand), and may specifically be any one or more selected from the group consisting of SCF, FLT3L, and a combination thereof, but are not limited thereto.

For the purpose of the present invention, the cytokines and growth factors are contained in the medium that induces direct cell-fate conversion of isolated cells into target cells, and the types of growth factors and cytokines are not particularly limited as long as they can be used to induce direct cell-fate conversion. As a specific embodiment, the cytokines and growth factors may be any one or more selected from the group consisting of IL-2, IL-15, IL-7, SCF, and FLT3L, but are not limited thereto.

In the present invention, the medium in b) may further contain any one or more selected from the group consisting of a GSK3β (glycogen synthase kinase 3β) inhibitor, a PDK1 (3-phosphoinositide-dependent kinase 1) inhibitor, and an AHR (aryl hydrocarbon receptor) inhibitor.

In the present invention, the term “GSK3β (glycogen synthase kinase-3β) inhibitor” refers to a substance that inhibits or suppresses the activity of GSK3β by directly/indirectly binding to the protein of GSK3β. The GSK3β inhibitor may be, for example, 1-azakenpaullone, 2-D08, 3F8, 5-bromoindole, 6-Bio, A 1070722, aloisine A, AR-A014418, alsterpaullone, AZD-1080, AZD2858, bikinin, BIO, BIO-acetoxime, bisindolylmaleimide I, bisindolylmaleimide I hydrochloride, CAS 556813-39-9, cazpaullone, CHIR98014, CHIR98023, CHIR99021(CT99021), CP21 R7, dibromocantherelline, GSK-3β inhibitor I, VI, VII, X, XI, XV, GSK-3 inhibitor IX, XVI, hymenidin, hymenialdisine, HMK-32, I3M (indirubin-3-monoxime, indirubin, indole-3-acetamide, IM-12, kenpaullone, L803-mts, leucettine L41, lithium, lithium carbonate, LY-2090314, manzamine A MeBIO, meridianineA, NP00111, NP031115, NP031111, NSC 693868, palinurin, Ro 31-8220 methanesulfonate, SB-216763, SB-415286, TC-G 24, TCS 2002, TCS 21311, Tideglusib, tricantin, trihydrochloride, tungstate, TWS-119, TZDZ-8, and zinc, and may be CHIR99021 (CT99021) as an example, but is not limited thereto.

In the present invention, the term “PDK1 (3-phosphoinositide-dependent kinase 1) inhibitor” refers to a substance that inhibits or suppresses the activity of PDK1 by directly/indirectly binding to the protein activity of PDK1. The PDK1 inhibitor may be BX-795, BX-912, PHT-427, GSK2334470, OSU-03012, and the like, and may be BX-795 as an example, but is not limited thereto.

In the present invention, the term “AHR (aryl hydrocarbon receptor) inhibitor” refers to a substance that downregulates or reduces the activity of AHR, a ligand-activated transcription factor activated by TCDD (dioxin(2,3,7,8-tetrachlorodibenzo-p-dioxin)). The AHR inhibitor may be, for example, StemRegenin 1 [SR1; 4-(2-((2-benzo[b]thiophen-3-yl)-9-isopropyl-9H-purin-6-yl)amino)ethyl)phenol hydrochloride] and CH-223191 (1-methyl-N-[2-methyl-4-[2-(2-methylphenyl)diazenyl]phenyl-1H-pyrazole-5-carboxamide), and may be StemRegenin 1 as an example.

However, the inhibitor is not limited to the description above as long as it plays a role in increasing the direct reprogramming efficiency.

In the present invention, the medium in b) may be divided into a first medium and a second medium.

For example, the first medium of the present invention may contain growth factors, cytokines, a GSK3β inhibitor, a PDK1 inhibitor, and/or an AHR inhibitor.

The first medium may contain SCF, FLT3L, IL-2, IL-7, IL-15, CT99021, and/or BX795, but is not limited thereto.

The first medium may further contain any one or more selected from the group consisting of FBS (fetal bovine serum), an antibiotic, and a combination thereof, but is not limited thereto.

The antibiotic may be penicillin/streptomycin, but is not limited thereto.

Specifically, the first medium in a) may contain FBS, penicillin/streptomycin, SCF, FLT3L, IL-2, IL-7, IL-15, CT99021, and/or BX795, but is not limited thereto.

More specifically, the first medium in a) may be StemSpan SFEM II containing 8% to 12% FBS, 0.1% to 2% penicillin/streptomycin, 10 to 30 ng/ml human SCF, 10 to 30 ng/ml human FLT3L, 150 to 250 IU/ml human IL-2, 10 to 30 ng/ml human IL-7, 10 to 30 ng/ml human IL-15, 2 to 4 μM CT99021, and 2 to 10 μM BX795, and may still more specifically be StemSpan SFEM II containing 9% to 11% FBS, 0.5% to 1.5% penicillin/streptomycin, 15 to 25 ng/ml human SCF, 15 to 25 ng/ml human FLT3L, 180 to 220 IU/ml human IL-2, 15 to 25 ng/ml human IL-7, 15 to 25 ng/ml of human IL-15, 2.5 to 3.5 μM CT99021, and/or 4 to 8 μM BX795, but is not limited thereto.

For example, the second medium of the present invention may contain growth factors, cytokines, and StemRegenin 1.

The second medium may contain SCF, FLT3L, IL-2, IL-7, IL-15, IL-21, and StemRegenin 1, but is not limited thereto.

The second medium may further contain any one or more selected from the group consisting of FBS, an antibiotic, and a combination thereof, but is not limited thereto.

The antibiotic may be penicillin/streptomycin, but is not limited thereto.

Specifically, the second medium in step a) may contain FBS, penicillin/streptomycin, SCF, FLT3L, IL-2, IL-7, IL-15, IL-21, and StemRegenin 1, but is not limited thereto.

More specifically, the second medium in a) may be StemSpan SFEM II containing 8% to 12% FBS, 0.1% to 2% penicillin/streptomycin, 10 to 30 ng/ml human SCF, 10 to 30 ng/ml human FLT3L, 150 to 250 IU/ml human IL-2, 10 to 30 ng/ml human IL-7, 10 to 30 ng/ml human IL-15, 10 to 30 ng/ml human IL-21, and 2 to 4 μM StemRegenin 1, and may still more specifically be StemSpan SFEM II containing 9% to 11% FBS, 0.5% to 1.5% penicillin/streptomycin, 15 to 25 ng/ml human SCF, 12 to 25 ng/ml human FLT3L, 180 to 220 IU/ml human IL-2, 15 to 25 ng/ml human IL-7, 15 to 25 ng/ml human IL-15, 15 to 25 ng/ml human IL-21, and 1.5 to 2.5 μM StemRegenin 1, but is not limited thereto.

The first and second media of the present invention can maximize NK cell preparation efficiency in preparing drNK cells through inhibition of BCL11B gene expression.

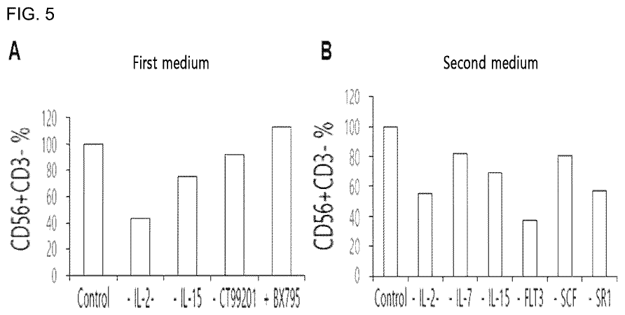

In an exemplary embodiment of the present invention, in order to examine the effect of the medium components of the first and second media on the preparation yield of shBCL11B-drNK, shBCL11B was transformed into PBMCs. Then the PBMCs were cultured in the first medium (positive control) or media each lacking one of human IL-2, human IL-15, or CHIR99021 among the components constituting the first medium or a medium further containing BX795 in addition to the components constituting the first medium, and then further cultured in the second medium, then the NK production efficiency was analyzed, and as a result, it was found that the shBCL11B-drNK was prepared at an efficiency of 43% in the cell population cultured in the medium lacking IL-2, 75% in the cell population cultured in the medium lacking IL-15, 92% in the cell population cultured in the medium lacking CHIR99021, and 113% in the cell population cultured in the medium further containing BX795 based on the efficiency (100%) in the positive control cultured in the first medium (FIG. 5A).

In another exemplary embodiment of the present invention, shBCL11B was transformed into PBMCs. Then the PBMCs were cultured in the first medium and further cultured in the second medium (positive control) or media each lacking one of 200 IU/ml human IL-2, 20 ng/ml human IL-7, 20 ng/ml human IL-15, 20 ng/ml human FLT3L, 20 ng/ml human SCF, or 2 μM SR1, then the NK production efficiency was analyzed, and as a result, it was found that the shBCL11B-drNK was prepared at an efficiency of 56% in the cell population cultured in the medium lacking IL-2, 69% in the cell population cultured in the medium lacking IL-15, 82% in the cell population cultured in the medium lacking IL-7, 81% in the cell population cultured in the medium lacking SCF, 38% in the cell population cultured in the medium lacking FLT3L, and 57% in the cell population cultured in the medium lacking SR1 based on the efficiency (100%) in the positive control cultured in the second medium (FIG. 5B).

Accordingly, it can be seen that the NK cell preparation efficiency can be improved using the first and second media of the present invention.

In the method, the isolated cells into which a direct cell-fate conversion factor is introduced may be cultured in the first medium in a) for 4 to 8 days and then cultured in the second medium in b) for 10 to 14 days, but are not limited thereto.

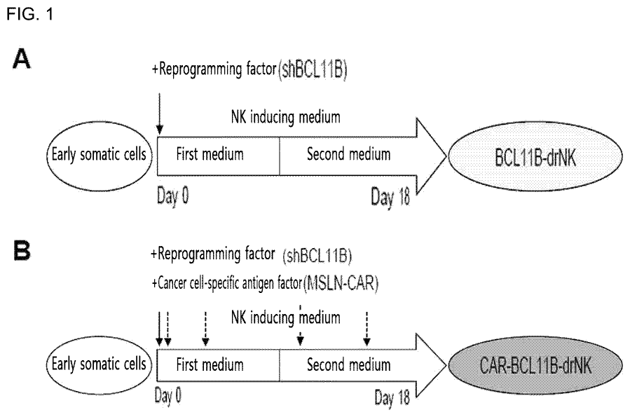

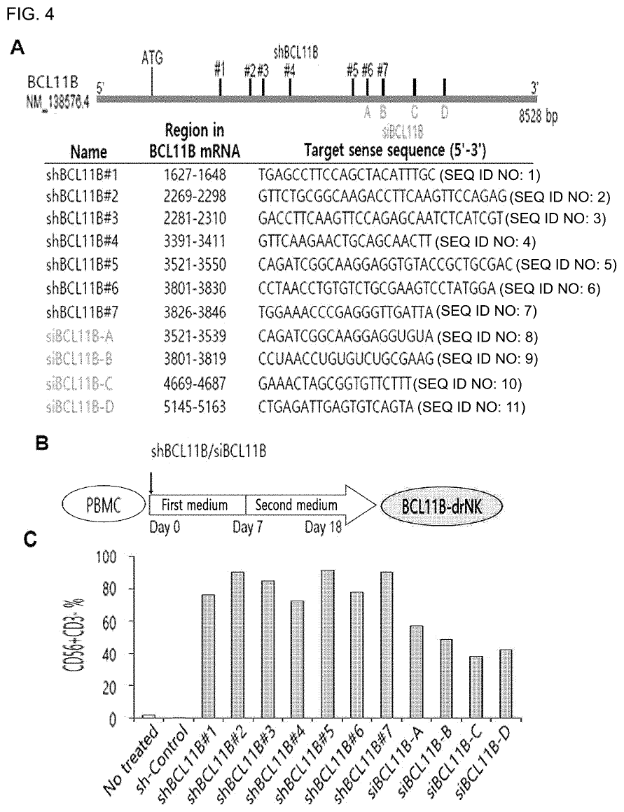

In an exemplary embodiment of the present invention, in order to introduce shRNA or siRNA against BCL11B as a reprogramming factor that induces cell-fate conversion into peripheral blood mononuclear cells (PBMC), PBMCs were treated with a lentivirus expressing BCL11B-specific shRNA or siRNA was introduced into PBMCs for transformation, and the PBMCs were cultured in the first medium until day 7, and then cultured in the second medium until day 18 to prepare direct cell-fate conversion induced drNK cells (shBCL11B-drNK) (FIGS. 1A and 4B).

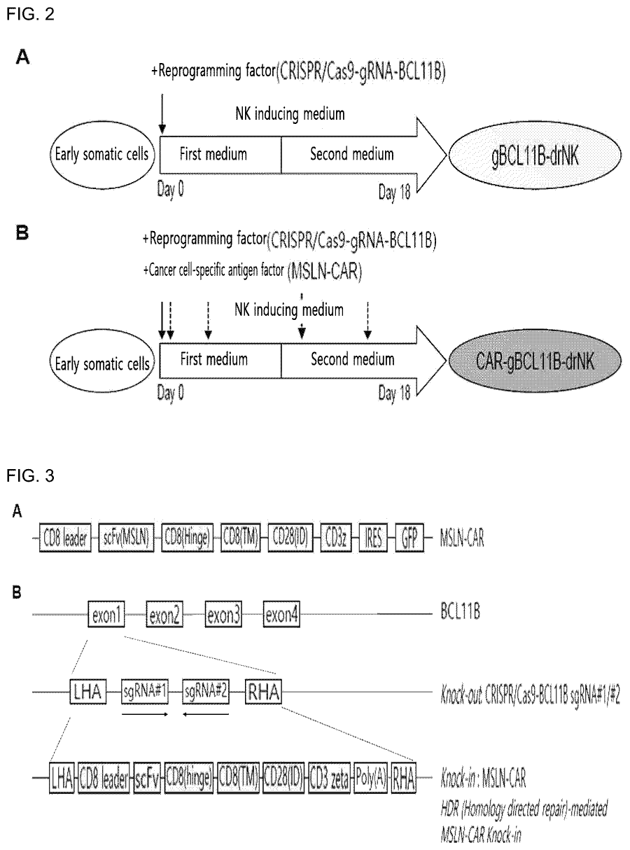

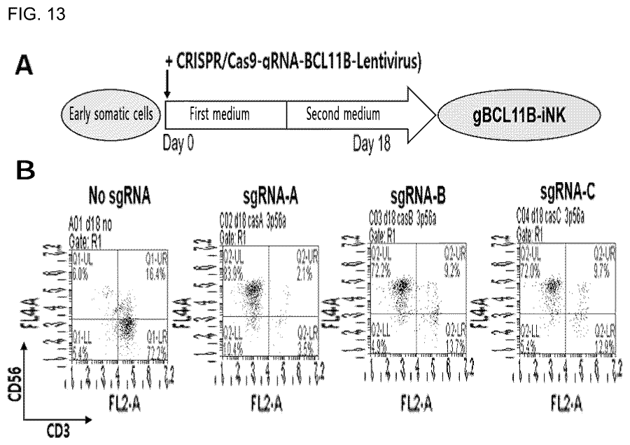

In another exemplary embodiment of the present invention, PBMC cells were treated with a lentivirus expressing a CRISPR/Cas9 vector containing sgRNA targeting BCL11B as a cell-fate conversion inducing factor for transformation. They were then cultured in the first medium until day 7, and subsequently cultured in the second medium until day 18 to prepare direct cell-fate conversion induced drNK cells (gBCL11B-drNK) (FIGS. 2A and 13A).

Another aspect of the present invention provides a CAR-drNK cell preparation method.

The terms used here are as described above.

The method for preparing a CAR-drNK cell may include additionally introducing a CAR gene in any one or more steps selected from a) or b) in the drNK cell preparation method of the present invention. As a specific embodiment, the additionally introduced CAR gene may be any one or more selected from the group consisting of CD19-CAR, MSLN-CAR, and HER2-CAR. Specifically, the CAR gene may be introduced into the BCL11B knock-out (KO) base sequence through knock-in (KI), but is not limited thereto.

In the present invention, the term “CAR gene” refers to a gene encoding a chimeric antigen receptor consisting of an extracellular domain, a transmembrane domain, and an intracellular domain by containing genes encoding the extracellular domain, transmembrane domain, and intracellular domain including an antibody domain (scFv). For the purposes of the present invention, the CAR gene of the present invention may be any one or more selected from the group consisting of a CD19-CAR gene containing CD19 scFv, an MSLN-CAR gene containing MSLN (mesothelin) scFv, and an HER2-CAR gene containing HER2 (human epidermal growth factor receptor 2) scFv, but is not limited thereto.

As CAR targeting factors for solid tumors, it is known that EGFRvIII (Morgan R A, Hum Gene Ther. 2012; 23:1043-1053), MUC-1 (Wilkie S, J Immunol. 2008; 180:4901-4909), MAGE (Willemsen R A, Gene Ther. 2001; 8:1601-1608), CEA (Emtage P C, Clin Cancer Res. 2008; 14:8112-8122), PSMA, GD2, CA125, Her2 and MSLN, FAP, VEGFR (Kakarla S, Cancer J. 2014; 20:151-155), and the like may be used.

The CD19 is a cluster of differentiation (CD) and is assigned the number 19 to identify cell surface molecules according to immunophenotype, and the CD19 refers to a marker of B lymphocytes. The CD19 is known to be expressed on most B-cell malignant cancer cells, providing an ideal target for these carcinomas.

As an example, the CAR gene may be any one or more selected from the group consisting of i) a CAR gene (CD19-CAR gene) containing CD8 leader, CD19 scFv, CD8 hinge, CD8 transmembrane domain (TM) and Fc-γ (gamma) receptor; ii) a CAR gene (MSLN-CAR gene) containing CD8 leader, MSLN (mesothelin) scFv, CD8 hinge, CD8 transmembrane domain, CD28 intracellular domain, CD3ζ and IRES; and iii) a CAR gene (HER2-CAR gene) containing CD8 leader, HER2 (human epidermal growth factor receptor 2) scFv, CD8 hinge, CD8 transmembrane domain, CD28 intracellular domain, CD3ζ and IRES, but is not limited thereto.

The CD8 leader may include the base sequence of SEQ ID NO: 14, the CD19 scFv of SEQ ID NO: 15, the MSLN scFv of SEQ ID NO: 16, the HER2 scFv of SEQ ID NO: 17, the CD8 hinge of SEQ ID NO: 18, the CD8 transmembrane domain of SEQ ID NO: 19, the Fc-γ receptor of SEQ ID NO: 20, the CD28 intracellular domain of SEQ ID NO: 21, CD3ζ of SEQ ID NO: 22, and the IRES inserted to clone the CAR gene into a vector constituting a double cistron of SEQ ID NO: 23, but are not limited thereto.

The CAR gene may further contain GFP (green fluorescent protein), but is not limited thereto.

The GFP may include the base sequence of SEQ ID NO: 24, but is not limited thereto.

The base sequences of SEQ ID NO: 14 to SEQ ID NO: 24 may be found in NCBI GenBank, a known database.

In the present invention, the base sequences of SEQ ID NO: 14 to SEQ ID NO: 24 may include base sequences having at least 70%, 80%, 85%, 90%, 95%, 96%, 97%, 98%, or 99% or more homology or identity with SEQ ID NO: 14 to SEQ ID NO: 24. It is obvious that base sequences having some sequences deleted, modified, substituted, or added are also included within the scope of the present invention as long as the base sequences have such homology or identity and exhibit functions corresponding to those of the base sequences of SEQ ID NO: 14 to SEQ ID NO: 24.

In the present invention, the terms “homology and identity” refer to the degree to which two given amino acid sequences or base sequences are related and may be expressed as a percentage. The terms homology and identity may often be used interchangeably.

Sequence homology or identity of conserved polynucleotides or polypeptides is determined by standard alignment algorithms, and default gap penalties established by the program being used may also be used. Substantially homologous or identical sequences can generally hybridize with each other in the entire sequence or at least about 50%, 60%, 70%, 80%, or 90% of the full length under moderate or high stringent conditions. In hybridization, polynucleotides containing degenerate codons instead of codons in polynucleotides are also contemplated.

The homology or identity of polypeptide or polynucleotide sequences can be determined using, for example, the literature algorithm BLAST [see: Karlin and Altschul, Pro. Natl. Acad. Sci. USA, 90, 5873(1993)] or FASTA by Pearson (see: Methods Enzymol., 183, 63, 1990). Based on this algorithm BLAST, a program called BLASTN or BLASTX has been developed (see: http://www.ncbi.nlm.nih.qov). Whether arbitrary amino acid or polynucleotide sequences have homology, similarity or identity can be determined by comparing the sequences by Southern hybridization experiments under defined stringent conditions, and suitable hybridization conditions to be defined are within the scope of the art and are well known to those skilled in the art (for example, J. Sambrook et al., Molecular cloning, A laboratory manual, 2nd Edition, Cold spring harbor laboratory press, Cold spring harbor, New York, 1989; F. M. Ausubel et al., Current protocols in molecular biology).

In the present invention, the CAR gene may be introduced into cells by the same method as the method of introducing a direct cell-fate conversion factor described above, and the direct cell-fate conversion factor and CAR gene may be introduced simultaneously or sequentially at the desired time under desired conditions.

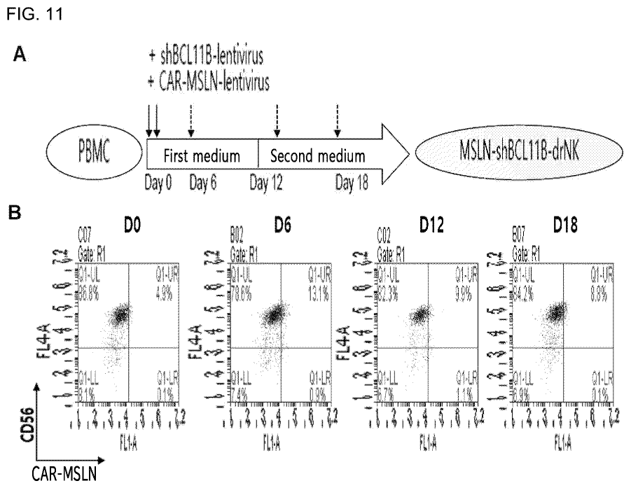

In an exemplary embodiment of the present invention, in order to introduce shRNA or siRNA against BCL11B as a reprogramming factor that induces cell-fate conversion into PBMC cells, PBMCs were treated with a lentivirus expressing BCL11B-specific shRNA or siRNA which was introduced into PBMCs for transformation, and were transformed with a lentivirus expressing the MSLN-CAR gene, and at this time, the PBMCs were cultured in the first medium and then further cultured in the second medium to prepare direct cell-fate conversion induced drNK cells (MSLN-shBCL11B-drNK) expressing the CAR (MSLN-CAR) gene (FIGS. 1B and 11A).

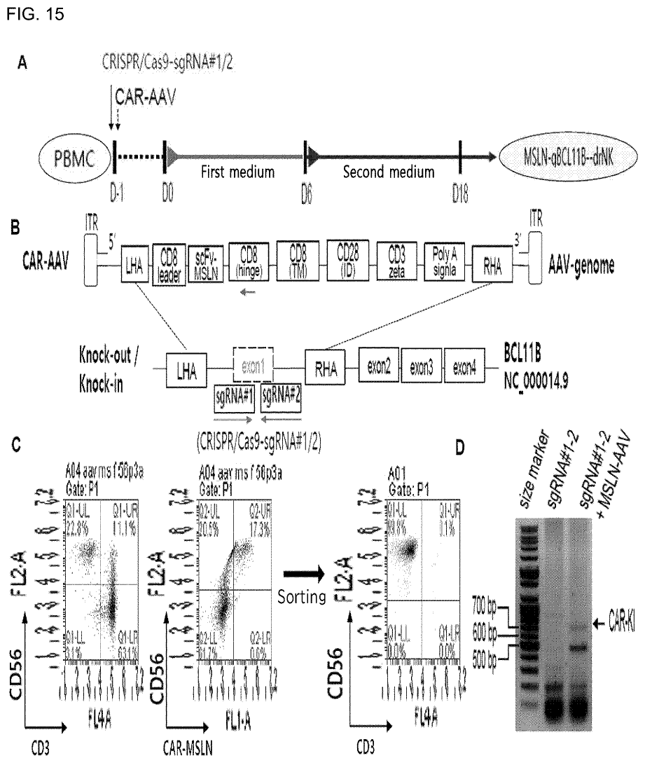

In another exemplary embodiment of the present invention, CAR-AAV as a donor plasmid in which the CAR coding sequence can be inserted and knocked-in (KI) into the cut site at the same time as BCL11B knock-out (KO) was introduced into PBMC cells for transformation, and the cells were treated with a lentivirus expressing a CRISPR/Cas9 vector containing sgRNA targeting BCL11B as a cell-fate conversion inducing factor for transformation, and at this time, the cells were cultured in the first medium and then further cultured in the second medium to prepare direct cell-fate conversion induced drNK cells (MSLN-gBCL11B-drNK) expressing the CAR (MSLN-CAR) gene (FIGS. 2B and 15).

Still another aspect of the present invention provides drNK cells prepared by the method of the present invention.

Still another aspect of the present invention provides CAR-drNK cells prepared by the method of the present invention.

The terms used here are as described above.

The drNK cells prepared by the method of the present invention may express any one or more selected from the group consisting of CD56⁺, CD3⁻, and a combination thereof, but are not limited thereto.

The “CD56⁺” and “CD3⁻” are indicators on the surface of NK cells, and in the present invention, whether NK cells were prepared was determined by analyzing the expression of CD56⁺, CD3⁻, and a combination thereof using a flow cytometer.

In an exemplary embodiment of the present invention, in order to examine the production and yield of shBCL11B-drNK cells, the cells were stained with a CD56 antibody and a CD3 antibody, then the NK cell population (CD56⁺ and CD3⁻) was analyzed using a flow cytometer, and as a result, it was found that CD56⁺CD3⁻ (shBCL11B-drNK) cells were prepared at an efficiency of 2% (No-treated) and 0% (sh-Control) in the controls, respectively, but at an efficiency of 73% to 92% in the shBCL11B#1, shBCL11B#2, shBCL11B#3, shBCL11B#4, shBCL11B#5, shBCL11B#6 and shBCL11B#7 treated groups, and at an efficiency of 38% to 57% in siBCL11B-A, siBCL11B-B, siBCL11B-C, and siBCL11B-D treated groups (FIG. 4C).

In another exemplary embodiment of the present invention, in order to examine the production and yield of gBCL11B-drNK cells, the cells were stained with a CD56 antibody and a CD3 antibody, then the NK cell populations (CD56⁺ and CD3⁻) were analyzed using a flow cytometer, and as a result, it was found that CD56⁺CD3⁻(gBCL11B-drNK) cells were prepared at an efficiency of 83.9%, 72.2%, and 72.0%, respectively, in the Cas9-sgRNA-BCL11B treated groups containing sgRNA-A, sgRNA-B or sgRNA-C compared to the No-treated group (6.0%) (FIG. 13B).

The CAR-drNK cells prepared by the method of the present invention may express any one or more selected from the group consisting of CD56⁺, CD3⁻, and a combination thereof, but are not limited thereto.

In an exemplary embodiment of the present invention, in order to examine the production and yield of MSLN-shBCL11B-drNK cells, the cells were stained with CD56 antibody and MSLN-CAR antigen, then the NK cell populations (CD56⁺ and MSLN-CAR⁺) were analyzed using a flow cytometer, and as a result, it was found that CD56⁺MSLN⁺(MSLN-shBCL11B-drNK) cells were prepared at an efficiency of 4.9% to 13.1% (FIG. 11B).

In another exemplary embodiment of the present invention, in order to examine the production and yield of MSLN-gBCL11B-drNK cells, the cells were stained with CD56 antibody, CD3 antibody, and MSLN-CAR antigen, then the drNK cell populations (CD56⁺, CD3⁻, and MSLN-CAR⁺) were analyzed using a flow cytometer, and as a result, it was found that the CD56⁺MSLN⁺ cell population was prepared at an efficiency of 17.3% compared to the CD56⁺CD3⁻ cell population (22.8%) (FIG. 15C).

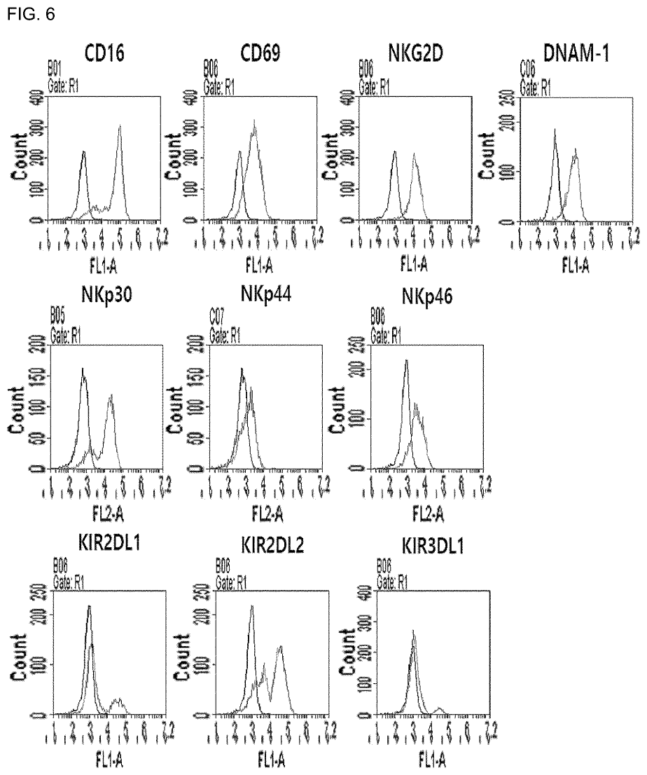

In still another exemplary embodiment of the present invention, the expression characteristics of NK-specific markers were verified, and as a result, it was found that in shBCL11B-drNK cells, MSLN-shBCL11B-drNK cells, and gBCL11B-drNK cells, activating receptors such as CD16, CD69, NKG2D, NKp30, NKp44, NKp46, and DNAM-1 were expressed at a higher frequency than inhibitory receptors such as KIR2DL1, KIR2DL2 and KIR3DL1, showing similar tendencies (FIGS. 6, 12, and 14).

Through this, it has been found that conversion from human blood cells into NK cells is possible through direct cell-fate conversion using shRNA, siRNA, or Cas9/sgRNA against BCL11B.

Still another aspect of the present invention provides a cell therapeutic composition for the prevention or treatment of cancer, containing the cells prepared by the method of the present invention as an active ingredient.

The terms used here are as described above.

In the present invention, the cancer may be a cancer associated with the expression of any one or more of CD19, MSLN, or HER2, and may specifically be a cancer that results in prevention or treatment by the immune response and the like of drNK cells and/or CAR-drNK cells. The cancer may be, for example, liver cancer, thyroid cancer, malignant tumor of thyroid gland, papillary thyroid cancer, medullary thyroid cancer, pseudomyxoma, intrahepatic biliary tract cancer, gamblastoma, tracheoblastoma, basal cell carcinoma, testicular cancer, bone marrow cancer, myeloma, myelodysplasia, osteosarcoma, osteogenic sarcoma, colon cancer, glioblastoma, oral cancer, lip cancer, mycosis fungoides, ovarian cancer, malignant ovarian germ cell tumor, male breast cancer, cystic cancer, endothelial sarcoma, brain cancer, meningioma, pituitary adenoma, biliary tract carcinoma, colorectal cancer, head and neck cancer, craniopharyngioma, biliary tract cancer, gallbladder cancer, multiple myeloma, lymphovascular endothelial sarcoma, lymphoma, Hodgkin's lymphoma, non-Hodgkin's lymphoma, lymphangiosarcoma, membranous carcinoma, retinoblastoma, choroidal melanoma, diffuse large B-cell lymphoma, bladder cancer, preleukemia, acute myeloid leukemia, acute lymphoblastic leukemia, B-cell acute lymphoblastic leukemia (BALL), acute leukemia, acute lymphoblastic leukemia (ALL), chronic leukemia, chronic myeloid leukemia (CML), chronic lymphocytic leukemia (CLL), T-cell acute lymphocytic leukemia (TALL), small lymphocytic leukemia (SLL), ampullary cancer of Vater, non-melanoma skin cancer, peritoneal cancer, parathyroid adenocarcinoma, adrenal cancer, sinonasal cancer, squamous cell cancer, non-small cell lung cancer, Wilms tumor, epithelial carcinoma, ependymoma, germ cell cancer, renal cancer, renal cell carcinoma, glioma, neuroblastoma, renal pelvic cancer, neuroblastoma, neuroendocrine tumor, duodenal cancer, tongue cancer, fibrosarcoma, adenocarcinoma, astrocytoma, small cell pineal cell tumor, pediatric brain tumor, pediatric lymphoma, small intestine cancer, meningioma, medulloblastoma, renal cell cancer, esophageal cancer, eye cancer, malignant soft tissue tumor, malignant air tumor, malignant lymphoma, malignant mesothelial tumor, malignant melanoma, eye tumor, vulvar cancer, Ewing chondrosarcoma cancer, ureteral cancer, urethral cancer, cancer of unknown primary site, sarcoma, carcinoid, breast cancer, choriocarcinoma, stomach cancer, gastric lymphoma, gastric carcinoid tumor, gastrointestinal stromal tumor, penile cancer, pharyngeal cancer, gestational trophoblastic disease, uterine cancer, cervical cancer, endometrial cancer, uterine sarcoma, prostate cancer, myxosarcoma, seminoma, liposarcoma, mesothelioma, rectal cancer, vaginal cancer, metastatic bone tumor, metastatic brain tumor, mediastinal cancer, spinal cord cancer, chordoma, acoustic neuroma, vestibular schwannoma, salivary gland cancer, pancreatobiliary cancer, pancreatic cancer, odontoma cancer, Kaposi's sarcoma, Paget's disease, tonsil cancer, sebaceous carcinoma, oligodendroglioma retinoblastoma, leiomyosarcoma, squamous cell carcinoma, squamous cell carcinoma, lung cancer, lung adenocarcinoma, lung squamous cell carcinoma, skin cancer, anal cancer, laryngeal cancer, pleural cancer, thymic cancer, hemangioblastoma, blood cancer, hemangiosarcoma, synovioma, rhabdomyosarcoma, melanoma, rare cancer, fetal carcinoma, sweat gland carcinoma, sarcoma, and combinations thereof, and may specifically be blood cancer, colorectal cancer, liver cancer, lung cancer, pancreatic cancer, brain cancer, ovarian cancer, breast cancer, prostate cancer, melanoma, myoma, renal cancer, adenocarcinoma, sarcoma, uterine cancer, stomach cancer, and bladder cancer, but is not limited thereto.

As an example, the cancer may be a cancer associated with the expression of MSLN. As another example, the cancer may be lymphoma, leukemia, myeloma, mesothelioma, uterine cancer, head and neck cancer, esophageal cancer, synovial sarcoma, renal cancer, transitional cell carcinoma, adenocarcinoma, germ cell tumor, bile duct cancer/cholangiocarcinoma, papillary serous adenocarcinoma, colorectal cancer, liver cancer, lung cancer, pancreatic cancer, glioblastoma, colon cancer, ovarian cancer, breast cancer, prostate cancer, melanoma, myoma, thyroid cancer, osteosarcoma, choriocarcinoma, stomach cancer, glioma, soft tissue sarcoma, neuroendocrine tumor, pituitary tumor, oligodendroglioma, gastrointestinal stromal tumor, gallbladder cancer, small intestine cancer, solitary fibroma, thymic cancer, and bladder cancer, but is not limited thereto.

In the present invention, the term “prevention” refers to any action that suppresses or delays the development of cancer by administration of the composition.

In the present invention, the term “treatment” refers to any action that improves or beneficially changes cancer symptoms by administration of the composition.

In the present invention, the term “cell therapeutic” refers to a medicine used for the purposes of treatment, diagnosis, and prevention, which is cells and tissues prepared through isolation from an individual, culture, and special manipulation, and refers to a medicine used for the purposes of treatment, diagnosis, and prevention through a series of actions such as proliferating and selecting living autologous, allogeneic, or xenogeneic cells in vitro or changing the biological characteristics of cells by other methods in order to restore the function of cells or tissues.

The cell therapeutic composition may be effective in preventing or treating cancer by containing the drNK cells and/or CAR-drNK cells prepared by the method of the present invention.

The cell therapeutic composition may contain the drNK cells and/or CAR-drNK cells at 1.0×10 to 1.0×10¹⁰ cells/ml, specifically 1.0×10⁶ to 1.0×10⁹ cells/ml based on the total weight of the composition, but is not limited thereto.

The cell therapeutic composition may be formulated and administered as a unit dosage pharmaceutical preparation suitable for administration into the patient's body according to a common method in the pharmaceutical field, and the preparation has an effective dosage by one time or several times of administration. As the formulation suitable for this purpose, parenteral preparations including injections such as ampoules for injection, infusions such as infusion bags, and sprays such as aerosol preparations may be preferred. The ampoule for injection may be prepared in mixture with an injection solution immediately before use, and physiological saline, glucose, mannitol, Ringer's solution, and the like may be used as the injection solution. As the infusion bag, those made of polyvinyl chloride or polyethylene may be used, and examples thereof include infusion bags manufactured by Baxter, Becton Dickinson, Medcep, National Hospital Products or Terumo.

In addition to the active ingredient, the pharmaceutical preparation may additionally contain one or more pharmaceutically acceptable common inert carriers, for example, a preservative, analgesic agent, solubilizer or stabilizer in the case of injections, and a base, excipient, lubricant or preservative in the case of preparations for topical administration.

The cell therapeutic composition of the present invention or pharmaceutical preparation thereof prepared in this manner may be administered together with or in the form of a mixture with other cells used in cancer treatment by an administration method commonly used in the art, and specifically can be engrafted or transplanted directly into the diseased area of a patient in need of treatment, or directly implanted or injected into the abdominal cavity, but is not limited thereto. As for the administration, both non-surgical administration using a catheter and surgical administration such as injection or transplantation after incision of the diseased area are possible. In addition to parenteral administration according to common methods, for example, direct administration to the lesion or transplantation by intravascular injection is also possible.

The cell therapeutic composition may be administered at a dose of 0.0001 to 1,000 mg/kg per day, specifically 0.01 to 100 mg/kg per day, and may be administered one time or several times a day. However, it should be understood that the actual dosage of the active ingredient should be determined in light of various related factors such as the disease to be treated, the severity of the disease, the route of administration, and the patient's weight, age, and sex. Accordingly, the dosage does not limit the scope of the present invention in any way.

The drNK cells and/or CAR-drNK cells prepared by the method of the present invention may have excellent killing ability against various cancer cells.

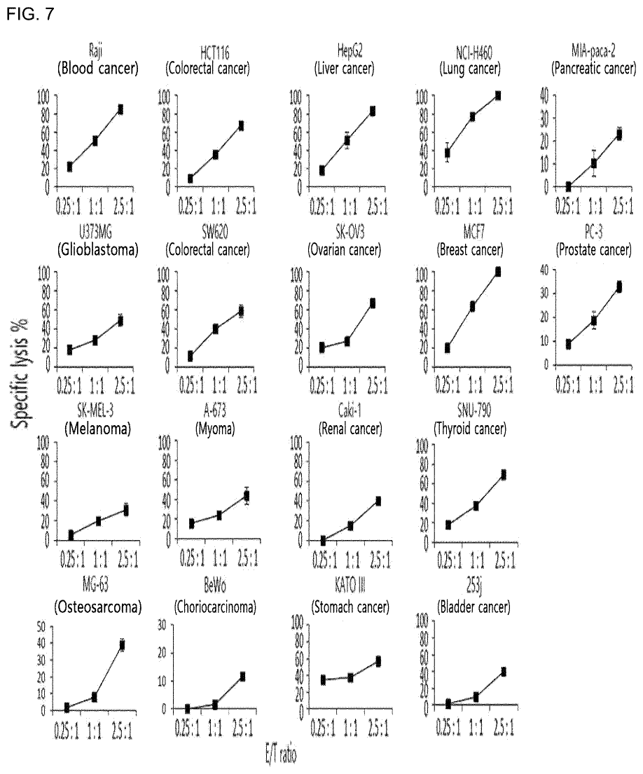

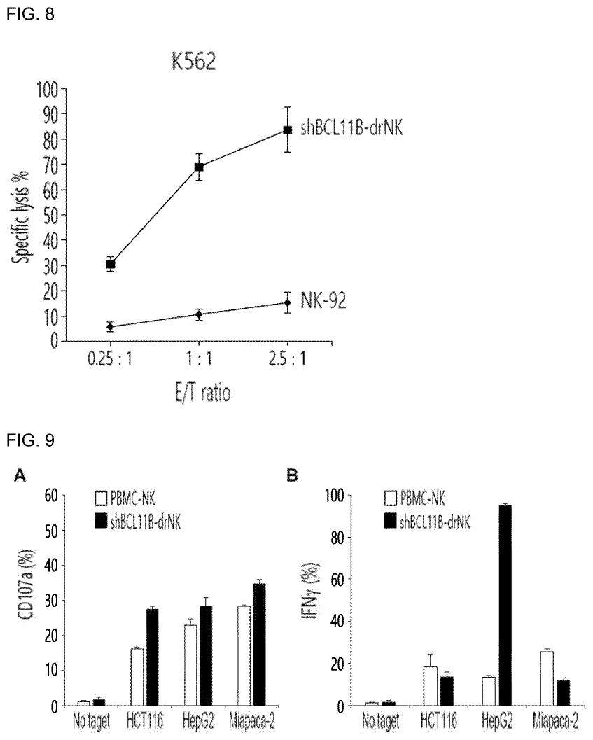

In an exemplary embodiment of the present invention, the cancer cell-killing ability of the constructed shBCL11B-drNK cells was examined, and as a result, it was found that the shBCL11B-drNK cells possess the ability to kill various types of cancer cells (lymphoma, colorectal cancer, liver cancer, lung cancer, pancreatic cancer, glioblastoma, colon cancer, ovarian cancer, breast cancer, prostate cancer, melanoma, myoma, thyroid cancer, osteosarcoma, choriocarcinoma, stomach cancer, and bladder cancer), and it was found that each shBCL11B-drNK cell exhibited high cancer cell-killing ability at low E (effector NK cell):T (target cancer cell) ratios (0.25:1 to 2.5:1) as well (FIG. 7). It was also found that shBCL11B-drNK cells were superior to NK-92 cells, which were existing human natural killer cells, in cancer cell-killing ability by about 5.2 to 5.5 times (FIG. 8).

In another exemplary embodiment of the present invention, PBMC-NK cells (NK derived from human peripheral blood mononuclear cells) were co-cultured with cancer cells, then expressed CD107a⁺ cells with cancer cell lytic ability were quantitatively analyzed, and as a result, it was found that the frequency (%) of CD107a⁺ cells increased in a case where shBCL11B-drNK cells and cancer cells were co-cultured compared to a case where PBMC-NK cells and cancer cells were co-cultured and the control (No target) not undergone co-culture (FIG. 9A). shBCL11B-drNK cells or PBMC-NK cells were co-cultured with cancer cells, then the number of IFN-gamma⁺ cells expressing cancer cell lytic IFN-gamma cytokine was quantitatively analyzed, and as a result, it was found that the frequency (%) of IFN-gamma⁺ cells increased compared to the control (No target) not undergone co-culture, and it was found that the frequency varied depending on the cancer cell type (FIG. 9B).

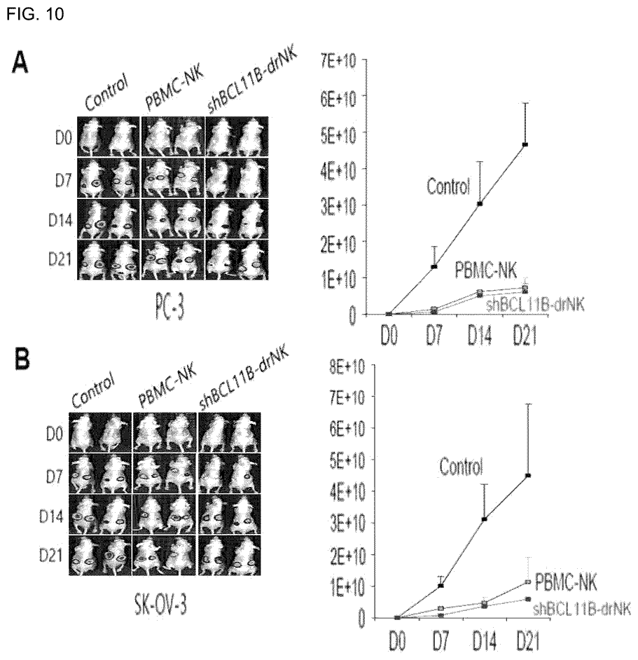

In still another exemplary embodiment of the present invention, PBMC-NK cells, shBCL11B-drNK cells, or PBS (phosphate-buffered saline) were injected into mouse prostate cancer models transplanted with PC-3, thereafter the tumor size in the PBMC-NK and shBCL11B-drNK administered groups was significantly reduced on day 21 compared to the tumor size in the control, and it was found that shBCL11B-drNK exhibited a superior anticancer effect compared to PBMC-NK (FIG. 10A). PBMC-NK cells, shBCL11B-drNK cells, or PBS were injected into mouse ovarian cancer models transplanted with SK-OV-3, thereafter, the tumor size was significantly reduced in the PBMC-NK and shBCL11B-drNK administered groups on day 21 compared to the tumor size in the control, and it was found that shBCL11B-drNK exhibited a superior anticancer effect compared to PBMC-NK (FIG. 10B).

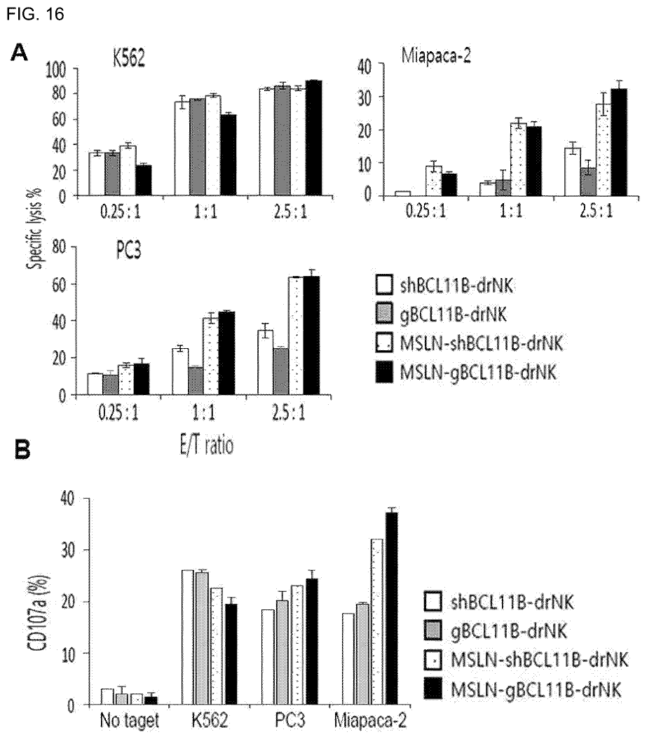

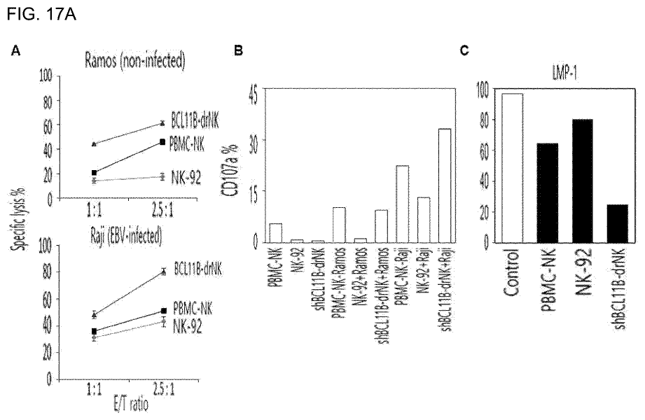

In still another exemplary embodiment of the present invention, shBCL11B-drNK cells, gBCL11B-drNK cells, MSLN-shBCL11B-drNK cells, and MSLN-gBCL11B-drNK cells were co-cultured with K562, which did not express MSLN, and PC-3 and Mia-paca-2, which expressed MSLN, then the cancer cell-killing ability was examined, and as a result, it was found that the four types of NK cells had similar cancer cell-killing ability against K562, which did not express MSLN, and CAR-drNK cells (MSLN-shBCL11B-drNK, MSLN-gBCL11B-drNK) had higher cancer cell-killing ability compared to non-CAR-drNK cells (shBCL11B-drNK, gBCL11B-drNK) against PC-3 and Mia-paca-2, which expressed MSLN, at low E:T ratios (0.25:1 to 2.5:1) as well (FIG. 16A).

shBCL11B-drNK cells, gBCL11B-drNK cells, MSLN-shBCL11B-drNK cells, and MSLN-gBCL11B-drNK cells were co-cultured with cancer cells, then the expressed CD107a⁺ cells were quantitatively analyzed, and as a result, it was found that the frequency (%) of CD107a⁺ cells increased in the CAR-drNK cells (MSLN-shBCL11B-drNK, MSLN-gBCL11B-drNK) compared to the non-CAR-drNK cells (shBCL11B-drNK, gBCL11B-drNK) against PC-3 and Mia-paca-2, which expressed MSLN (FIG. 16B).

Accordingly, the drNK cells and/or CAR-drNK cells prepared by the method of the present invention have excellent cancer cell-killing ability, excellent frequencies of CD107a⁺ cells and IFN-gamma⁺ cells after co-culture with cancer cells, and an excellent antitumor effect, and thus it can be seen that the drNK cells and/or CAR-drNK cells have an anticancer effect.

Another aspect of the present invention provides a pharmaceutical composition for the treatment or prevention of cancer, containing the cells prepared by the method of the present invention as an active ingredient.

The terms used here are as described above.

The pharmaceutical composition may be effective in preventing or treating cancer by containing the drNK cells and/or CAR-drNK cells prepared by the method of the present invention.

The pharmaceutical composition may contain the drNK cells and/or CAR-drNK cells at 1.0×10⁴ to 1.0×10¹⁰ cells/ml, specifically 1.0×10⁶ to 1.0×10⁹ cells/ml based on the total weight of the composition, but is not limited thereto.

The pharmaceutical composition may further contain pharmaceutically acceptable carriers, excipients, or diluents commonly used in the preparation of pharmaceutical compositions, and the carriers may include non-naturally occurring carriers. The carriers, excipients and diluents include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil.

The pharmaceutical composition may be formulated and used in the form of a tablet, pill, powder, granule, capsule, suspension, oral solution, emulsion, syrup, sterilized aqueous solution, non-aqueous solution, suspension, freeze-dried preparation, transdermal absorbent, gel, lotion, ointment, cream, patch, cataplasma, paste, spray, skin emulsion, skin suspension, transdermal patch, drug-containing bandage, or suppository according to a conventional method.

Specifically, in the case of being formulated, the formulation may be prepared using commonly used diluents or excipients such as fillers, weighting agents, binders, wetting agents, disintegrants, and surfactants. Solid preparations for oral administration include, but are not limited to, tablets, pills, powders, granules, capsules, and the like. These solid preparations may be prepared by mixing at least one or more excipients, for example, starch, calcium carbonate, sucrose, lactose, and gelatin. In addition to simple excipients, lubricants such as magnesium stearate and talc may also be used. In addition to liquids for oral administration and liquid paraffin, the solid preparations may be prepared by adding various excipients, for example, wetting agents, sweeteners, fragrances, and preservatives. Preparations for parenteral administration include sterile aqueous solutions, non-aqueous solutions, suspensions, emulsions, freeze-dried preparations and suppositories. As non-aqueous solvents and suspending agents, propylene glycol, polyethylene glycol, vegetable oil such as olive oil, injectable ester such as ethyl oleate, and the like may be used. As a base for suppositories, Witepsol, Macrogol, Tween 61, cacao, laurin, glycerogelatin, and the like may be used.

The pharmaceutical composition of the present invention may be administered in a pharmaceutically effective amount. The term “pharmaceutically effective amount” means an amount sufficient to treat a disease with a reasonable benefit/risk ratio applicable to medical treatment, and the effective dose level may be determined based on factors including the type and severity of the subject, age, sex, activity of the drug, sensitivity to the drug, time of administration, route of administration and rate of excretion, duration of treatment, and concurrently used drugs, and other factors well known in the medical field. For example, the pharmaceutical composition may be administered at a dose of 0.0001 to 1,000 mg/kg per day, specifically 0.01 to 100 mg/kg per day, and may be administered one time or several times a day.

The pharmaceutical composition may be administered as an individual therapeutic or in combination with other therapeutics, and may be administered sequentially or simultaneously with conventional therapeutics. The pharmaceutical composition may be administered singly or multiple times. It is important to administer the pharmaceutical composition in a minimum amount in which the maximum effect can be achieved without side effects, taking into account all of the above factors, and the amount may be easily determined by those skilled in the art.

The term “administration” means introducing the composition of the present invention into a subject by any suitable method, and the composition may be administered through any general route as long as it can reach the target tissue. The administration may be, but is not limited to, intraperitoneal administration, intravenous administration, intramuscular administration, subcutaneous administration, intradermal administration, oral administration, topical administration, and intranasal administration.

The term “subject” refers to all animals, including humans, monkeys, cows, horses, sheep, pigs, chickens, turkeys, quails, cats, dogs, mice, rats, rabbits or guinea pigs, that have developed or may develop cancer. The subject may include any type without limitation as long as the disease can be effectively prevented or treated by administering the pharmaceutical composition of the present invention to the subject.

Still another aspect of the present invention provides a method for treating cancer, the method including administering the cell therapeutic composition or pharmaceutical composition to a subject.

The terms used here are as described above.

Still another aspect of the present invention provides a cell therapeutic composition for the prevention or treatment of infectious diseases and/or inflammatory diseases, containing the cells prepared by the method of the present invention as an active ingredient.

The terms used here are as described above.

In the present invention, the infectious diseases may be caused or developed by any one or more selected from the group consisting of viruses, bacteria, and fungi, but are not limited thereto.

The viruses causing the infectious diseases may be RNA viruses and/or DNA viruses, but are not limited as long as they are viruses known in the art.

For example, the viruses may be Aviadenovirus, Alphatorquevirus, Arenavirus, Alphapapillomavirus, Adenovirus (Ad5), Astrovirus, Aichivirus, Amaparivirus, Aravanvirus, Auravirus, Australian bat lyssavirus, Bannavirus, Barmah Forest virus, Batken virus, Bunyamwera virus, Bunga virus, Bunyavirus, La Crosse virus, BK virus, BK polyoma virus, Cercopithecine herpesvirus, Cardiovirus, Crimean-congo hemorrhagic fever virus, Chapare virus, Chandipura virus, Chandipura vesiculovirus, Chikungunya virus, Cosavirus, Cowpox virus, Coxsackie virus, Coronavirus, Coronavirus alpha (beta, gamma, delta), Coltivirus, Cytomegalovirus, Densovirus, Dependovirus, Dependoparvovirus, Dengue virus, Deltaretrovirus, Dhori virus, Dugbe virus, Duvenhage virus, Eastern equine encephalitis virus, Ebolavirus, Echovirus, Ectromelia virus, ElBovirus, Erbovirus, Enterovirus, Encephalomyocarditis virus, Epstein-Barr virus (EBV), European bat lyssa (European bat lyssa) virus, Erythro virus, Flavivirus, Flexal virus, Fowl plague virus, GB virus C/Hepatitis G virus, Guanarito virus, Hantaan/Hantann river virus, Hanta virus, Hendra virus, Hepato/Hepatitis virus, Hepatitis virus (A, B, C, D, E), Horsepox virus, Human herpesvirus (HHV-6, HHV-7), Henipa virus, Herpes simplex virus (1, 2), Hepaci virus, Hepe virus, Henipa virus, Human immunodeficiency virus (HIV-1), Human cytomegalo virus, Ippy virus, Influenza virus (A, B, C), Isfahan virus, John cunningham (JC) virus, JC polyoma virus, Japanese encephalitis virus, Junin virus Junin arena virus, Kaposi's sarcoma-associated herpesvirus (KSHV), KI polyoma virus, Kobu virus, Kunjin virus, Lenti virus, Latino virus, Lassa virus, Lagos Bat virus, Lake victoria Marburg virus, Lyssa virus, Langat virus, Lordsdale virus, Louping ill virus, Lugo virus, Lymphocytic choriomeningitis virus, Lymphocryptovirus, Machupo virus, Mastadenovirus, Mamastrovirus, Machupo virus, Mayaro virus, MERS coronavirus, Measles virus, Marburg virus, Mumps virus, Mengovirus, Merkel cell polyoma virus, Mokola virus, Molluscum contagiosum virus, Mopeia virus, Monkeypox virus, Mobala virus, Morbili virus, Mupapilloma virus, Molluscipox virus, Murray valley encephalitis virus, New York virus, Nairovirus, Nipah virus, Norovirus, Norwalk virus, Oliveros virus, O'nyong-nyong virus, Orbivirus, Orthopoxvirus, Orthobunyavirus, Orthohepadnavirus, Orf virus, Oropouche virus, Orthomyxovirus, Orthopoxvirus, Orthopneumovirus, Papillomavirus, Papovavirus, Parainfluenza virus, Parechovirus, Pegivirus, Parana virus, Pichinde virus, Pirital virus, Parvovirus, Poliovirus, Polyomavirus, Poxvirus, Punta toro phlebovirus, Puumala virus, Phlebovirus, Roseolovirus, Rabies virus, Rhinovirus, Respiratory syncytial virus, Rift valley fever virus, Rhadinovirus, Rosa virus, Rubivirus, Ross river virus, Rotavirus, Rubella virus, Rubula virus, Respirovirus, Sabia virus, Sapovirus, Sagiyama virus, Salivirus, Sandfly fever Sicilian virus, Snowshoe hare virus, Sicilian phlebovirus, Spumaretrovirus, Sapporo virus, SARS (severe acute respiratory syndrome) virus, SARS coronavirus, SARS-CoV-2 virus, Semliki forest virus, Seoul virus, Simian foamy virus, Seadorna (‘South eastern Asia dodeca RNA) virus, Simian virus, Sindbis virus, Spuma virus, Southampton virus, St. Louis encephalitis (St. Louis encephalitis) virus, Tacaribe virus, Tamiami virus, Tick-borne Powassan virus, Tick-borne encephalitis virus, T-lymphotropic virus, Torovirus, Torque teno virus, Thogotovirus, Toscana virus, Uukuniemi virus, Vaccinia virus, Varicella-zoster virus, Variola virus, Venezuelan equine encephalitis virus, Vesicular stomatitis virus, Western equine encephalitis virus, West nile virus, Whitewater Arroyo virus, WU polyoma virus, Yaba monkey tumor virus, Yaba-like disease virus, Yellow fever virus, Vesiculovirus, Varicellovirus and Zika virus, but are not limited thereto.

Specifically, the viruses may be Epstein-Barr virus (EBV), hepatitis virus, human immunodeficiency virus (HIV), influenza virus, papilloma virus, SARS (severe acute respiratory syndrome) virus, SARS coronavirus, and SARS-CoV-2 virus, but are not limited thereto.

Bacteria that cause the infectious diseases may be Gram-negative bacteria and/or Gram-positive bacteria, but are not limited as long as they are bacteria known in the art.

As an example, the bacteria may be Achromobacter species (SPP), Acinetobacter spp., Actinomyces spp., Aeromonas spp., Alternaria spp., Anthrax spp., Aspergillus spp., Bacillus spp., Bacteroides spp., Bartonella spp., Brucella spp., Borrelia, spp., Bordetella spp., Burkholderia spp., Campylobacter spp., Capnocytophaga spp., Chlamydophila spp., Chlamydia spp., Citrobacter spp., Clostridium spp., Corynebacterium spp., Coxiella spp., Diphtheria spp., Ehrlichia spp., Escherichia spp., Enterobacter spp., Enterococcus spp., Erysipelothrix spp., Eikenella spp., Erwinia spp., Francisella spp., Fusobacterium spp., Gardnerella spp., Haemophilus spp., Helicobacter spp., Klebsiella spp., Lactobacillus spp., Legionella spp., Listeria spp., Leptospira spp., Micrococcus spp., Moraxella spp, Morganella spp., Moniliformis spp., Meningococcus spp., Mycobacterium spp., Mycoplasma spp., Neisseria spp., Nocardia spp., Pertussis spp., Pneumococcus spp., Pseudomonas spp., Pasteurella spp., Peptostreptococcus spp., Photorhabdus rhabdus spp., Porphyromonas spp., Propionibacterium spp., Proteus spp., Providencia spp., Pseudomonas spp., Salmonella spp., Serratia spp., Spiroplasma spp., Shigella spp., Staphylococcus spp., Stenotrophomonas spp., Streptococcus spp., Treponema spp., Vibrio spp., Wolbachia spp., Xenorhabdus spp., and Yersinia spp., but are not limited thereto.

For example, the bacteria may be Achromobacter xylosoxidans, Acinetobacter baumannii, Acinetobacter haemolyticus, Acinetobacter junil, Acinetobacter johnsonil, Actinomyces israeli, Aeromonas hydrophilia, Aeromonas veronol, Aspergillus fumigatus, Bacillus anthracis, Bacillus cereus, Bacillus thuringiensis, Bacteroides fragilis, Bacteroides melaninogenicus, Bartonella chomelii, Brucella abortus, Brucella canis, Brucella melitensis, Brucella microti, Brucella suis, Borrelia afzelii, Borrelia burgdorferi, Borrelia garinii, Borrelia recurrentis, Bordetella pertussis, Burkholderia cepacia, Burkholderia mimosarum, Burkholderia thailandensis, Campylobacter jejuni, Chlamydophila pneumoniae, Chlamydophila psittaci, Chlamydia pneumoniae, Chlamydia trachomatis, Clostridium acidisoli, Clostridium aciditolerans, Clostridium bartlettii, Clostridium botulinum, Clostridium cellobioparum, Clostridium cellulovorans, Clostridium citroniae, Clostridium clariflavum, Clostridium cocleatum, Clostridium difficile, Clostridium hiranonis, Clostridium irregular, Clostridium perfringens, Clostridium return, Clostridium sulfidigenes, Clostridium tetani, Clostridium thermobutyricum, Corynebacterium appendics, Corynebacterium callunae, Corynebacterium diphtheria, Coxiella burnetii, Ehrlichia canis, Ehrlichia chaffeensis, Escherichia coli, Escherichia albertii, Enterobacter cloacae, Enterococcus aecalis, Enterococcus faecalis, Enterococcus faecium, Erysipelothrix inopinata, Erysipelothrix rhusiopathiae, Erysipelothrix rhusiopathiae, Eikenella corrodens, Erwinia carotovora, Francisella tularensis, Fusobacterium necrophorum, Gardnerella vaginalis, Haemophilus influenza, Helicobacter brantae, Helicobacter pylori, Klebsiella pneumoniae, Lactobacillus plantarum, Legionella brunensis, Legionella drancourtii, Legionella drozanskil, Legionella impletisoli, Legionella pneumophila, Listeria monocytogenes, Leptospira alexanderi, Leptospira meyeri, Leptospira wolbachil, Micrococcus luteus, Moraxella catarrhalis, Moniliformis streptococcus, Mycobacterium abscessus, Mycobacterium aurum, Mycobacterium brumae, Mycobacterium farcinogenes, Mycobacterium fortuitum, Mycobacterium leprae, Mycobacterium marinum, Mycobacterium moriokaense, Mycobacterium pallens, Mycobacterium pneumoniae, Mycobacterium smegmatis, Mycobacterium tuberculosis, Mycobacterium tusciae, Mycoplasma pneumonia, Neisseria gonorrhoeae, Neisseria meningitides, Nocardia asteroids, Nocardia nova, Pseudomonas aeruginosa, Pasteurella multocida, Photorhabdus luminescens, Porphyromonas gingivalis, Propionibacterium acnes, Pseudomonas aeruginosa, Pseudomonas entomophila, Rickettsia aeschlimannii, Rickettsia asiatica, Rickettsia canadensis, Rickettsia montanensis, Rickettsia raoultii, Rickettsia, Salmonella enterica, Salmonella enteritis, Salmonella typhi, Salmonella typhimurium, Serratia liquefaciens, Serratia marcescens, Spiroplasma poulsonii, Shigella sonnei, Shigella dysenteriae, Staphylococcus aureus, Staphylococcus epidermidis, Staphylococcus haemolyticus, Staphylococcus hominis, Staphylococcus saprophyticus, Staphylococcus xylosus, Stenotrophomonas maltophilia, Streptococcus agalactiae, Streptococcus anginosus group, Streptococcus pneumoniae, Streptococcus pseudopneumoniae, Streptococcus pyogenes(groups A, B, C, G, F), Streptococcus viridans, Treponema azotonutricium, Treponema berlinense, Treponema denticola, Treponema medium, Treponema pallidum, Treponema primitia, Vibrio cholera, Xenorhabdus nematophila, Yersinia pseudotuberculosis, and Yersinia pestis, but are not limited thereto.

Specifically, the bacteria may be bacteria belonging to the genus Escherichia, the genus Streptococcus, and the like, more specifically may be E. coli, S. pseudopneumoniae, and the like, but are not limited thereto. The E. coli may include Enterotoxigenic E. coli, Enteropathogenic E. coli, Enteroinvasive E. coli, Enterohemorrhagic E. coli, and the like, but is not limited thereto.

As an example, the fungi that cause the infectious diseases may be Absidia spp., Alternaria spp., Aspergillus spp., Ascosphaera spp., Ajellomyces spp., Alternaria spp., Basidiobolus spp., Basidiomycete spp., Bipolaris Polaris spp., Blastomyces spp., Batrachochytrium spp., Beauveria spp., Bjerkandera spp., Botrytis spp., Blumeria spp., Candida spp., Coprinus spp., Chromoblastomycosis blastomycosis) spp., Cladosporium spp., Cladophialphora spp., Chaeotomium spp., Conidiobolus spp. Coccidioides spp., Colletotrichum spp., Cordyceps spp., Cryptococcus spp., Cunninghamella spp., Curvularia spp., Dactylaria spp., Dacrymyces spp., Epidermophyton spp., Exophiala spp., Fusarium spp., Geotrichum spp., Geomyces spp., Histoplasma spp., Lacazia spp., Lasiodiplodia spp., Leptosphaeria spp., Lomentospora spp., Malassezia spp., Madurella spp., Malassezia spp., Magnaporthe spp., Metarhizium spp., Microsporum spp., Mycosphaerella spp., Memnoniella spp., Melampsora spp., Mucor spp., Mucorales spp., Mucormycetes (Zygomycetes) spp., Myrothecium spp., Nectria spp., Paracoccidioides spp., Penicillium spp., Pneumocystis spp., Pneumocystis spp., Puccinia spp., Pseudoallescheria spp., Pythium spp., Ramichloridium spp., Rhizopus spp., Rhizoctonia spp., Saccharomyces spp., Scedosporium spp., Scedosporium spp., Schizophyllum spp., Sclerotinia spp., Scopulariopsis spp., Scytalidium spp., Stachybotrys spp., Sporotrix spp., Septoria spp., Syncephalastrum spp., Talaromyces spp., Tremella spp., Trichophyton spp., Trichosporon spp., Trichoderma spp., Ulocladium spp., Ustilago spp., and Zymoseptoria spp., but are not limited thereto.

For example, the fungi may be Absidia corymbifera, Alternaria alternate, Aspergillus alternate, Aspergillus versicolor, Aspergillus fumigatus, Aspergillus flavus, Aspergillus niger, Ajellomyces dermatitidis, Basidiobolus ranarum, Bipolaris spicifera, Blastomyces dermatitidis, Blastomyces gilchristii, Batrachochytrium dendrobatidis, Beauveria bassiana, Botrytis cinerea, Blumeria graminis, Candida albicans, Candida auris, Candida glabrata, Candida krusei, Candida lusitaniae, Candida parapsilosis, Candida krusei, Candida silvativa, Candida tropicalis, Coprinus cinereus, Cladosporium herbarum, Cladosporium immitis, Cladophialphora bantianum, Conidiobolus coronatus, Conidiobolus incongruus, Coccidioides immitis, Coccidioides posadasii, Cryptococcus gattii, Cryptococcus neoformans, Cunninghamella bertholletiae, Curvularia lunata, Exophiala jeanselmei, Exophiala dermatitidis, Fusarium graminearum, Fusarium moniliforme, Fusarium oxysporum, Fusarium solani, Dactylaria gallopava, Geotrichum candidum, Geomyces destructans, Histoplasma capsulatum, Lacazia loboi, Lomentospora prolificans, Ma/assezia furfur, Magnaporthe oryzae, Metarhizium anisopliae, Mycosphaerella graminicola, Melampsora lini, Mucor circillenoides, Mucor mucedo, Mucor pusillus, Paracoccidioides brasiliensi, Paracoccidioides lutzii, Penicillium brevicompactum, Penicillium chrysogenum, Penicillium citrinum, Penicillium corylophilum, Penicillium cyclopium, Penicillium expansum. Penicillium fellutanum, Penicillium marneffei, Penicillium spinulosum, Penicillium viridicatum, Pneumocystis carinii, Pneumocystis jirovecii, Pneumocystis murina, Pseudoallescheria boydii, Pythium debaryanum, Rhizopus oryzae, Rhizoctonia solani, Saccharomyces cerevisiae, Scedosporium anamorphs, Scedosporium apiospermum, Scedosporium prolificans, Schizophyllum commune, Sclerotinia americana, Stachybotrys chartarum, Sporothrix schenckii, Septoria tritici, Talaromyces marneffei, Trichophyton rubrum, Trichophyton interdigitale, Trichophyton purpureum, Trichophyton violaceum, Trichosporon asahii, Trichoderma lignorum, Trichoderma viride, Ustilago maydis, and Zymoseptoria tritici, but are not limited thereto.

Specifically, the fungi may be fungi belonging to the genus Aspergillus, the genus Candida, the genus Absidia, the genus Mucor, the genus Rhizopus, and the like, and more specifically may be Candida albicans, Aspergillus fumigatus, Absidia corymbifera, Mucor circillenoides, Mucor mucedo, Mucor pusillus, Rhizopus oryzae, and the like, but are not limited thereto.

The composition of the present invention may be effective in preventing or treating infectious diseases and/or inflammatory diseases caused by the infectious diseases by containing the drNK cells and/or CAR-drNK cells prepared by the method of the present invention.

The drNK cells and/or CAR-drNK cells prepared by the method of the present invention can exhibit antiviral effects against various viruses.

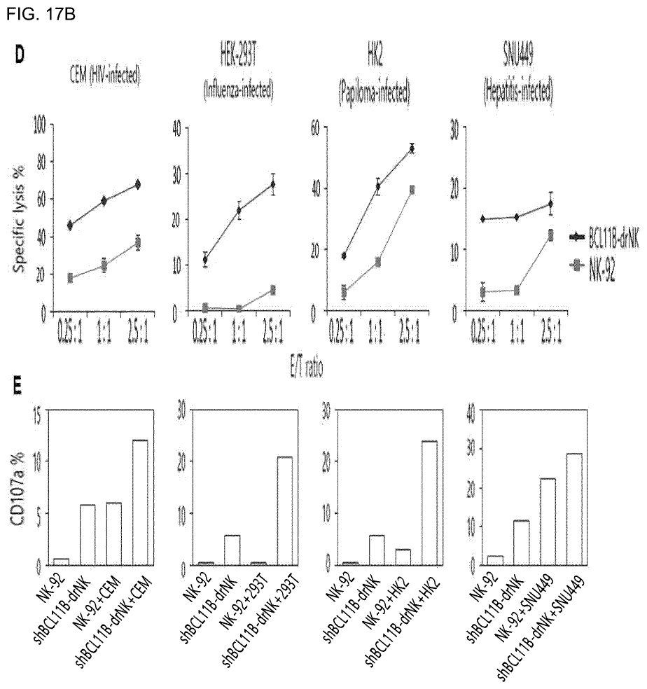

In an exemplary embodiment of the present invention, in order to examine the antiviral effect of BCL11B-drNK, the cell-killing ability against virus-uninfected B-lymphoma Ramos and EBV-infected B-lymphoma Raji cells and CD107a⁺ cells were measured, and as a result, it was found that BCL11B-drNK had higher toxicity against EBV-infected Raji cells than Ramos compared to the control cells NK-92 and PBMC-NK (FIGS. 17A and 17B).

Raji cells containing EBV were transformed to express GFP using lentivirus to obtain GFP-Raji cells, then GFP-Raji and NK cells were co-cultured, then the expression level of LMP-1 (latent membrane protein 1), an EBV-specific gene, was measured, and as a result, it was found that the expression level of LMP-1 decreased compared to that of GFP in a case where NK cells and GFP-Raji cells were co-cultured compared to the control not undergone co-culture, and it was found that the degree of decrease in LMP-1 expression was higher in shBCL11B-drNK cells than in the control cells NK-92 and PBMC (FIG. 17C).

In another exemplary embodiment of the present invention, it was found that drNK cells exhibited higher killing ability against CEM T cells, HEK-293T cells, HK2 proximal tubule cells, and SNU449 liver cells, which were each infected with human immunodeficiency virus (HIV), influenza virus, papilloma virus, and hepatitis virus, compared to a control cell NK-92 (FIG. 17D) and a higher frequency of CD107a⁺ cell expression (FIG. 17E) at a low E:T ratio as well.

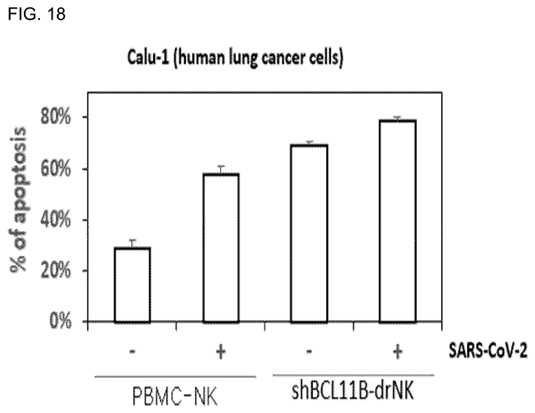

In still another exemplary embodiment of the present invention, it was found that co-culture with shBCL11B-drNK cells increased the apoptosis of cells infected with the SARS-CoV-2 virus compared to the positive control (PMBC-NK cells) (FIG. 18).

Through this, it has been found that shBCL11B-drNK cells exhibit more significant antibacterial efficacy against RNA viruses and DNA viruses compared to PBMC-NK cells and NK-92 cells.

The drNK cells and/or CAR-drNK cells prepared by the method of the present invention can exhibit antibacterial effects against various bacteria.

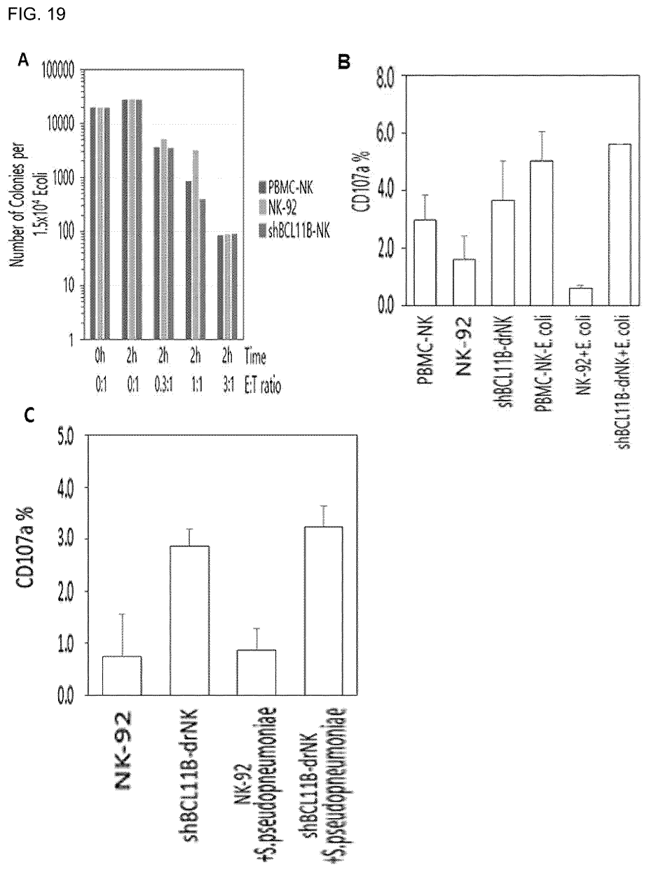

In an exemplary embodiment of the present invention, the effect of killing E. coli, a Gram-negative bacterium, of drNK cells was examined through the number of E. coli colonies (FIG. 19A) and the expressed CD107a⁺ cell frequency (FIG. 19B) after co-culture, and as a result, it was found that drNK exhibited higher antibacterial efficacy compared to NK-92 and PBMC-NK cells.

In another exemplary embodiment of the present invention, the antibacterial effect of drNK cells against Streptococcus, a Gram-positive bacterium, was examined through the expressed CD107a⁺ cell frequency after co-culture, and as a result, it was found that drNK exhibited higher antibacterial efficacy compared to NK-92 cells (FIG. 19C).

Through this, it has been found that shBCL11B-drNK cells exhibit more significant antibacterial efficacy against Gram-negative bacteria and Gram-positive bacteria compared to PBMC-NK cells and NK-92 cells.

The drNK cells and/or CAR-drNK cells prepared by the method of the present invention can exhibit antifungal effects against various fungi.

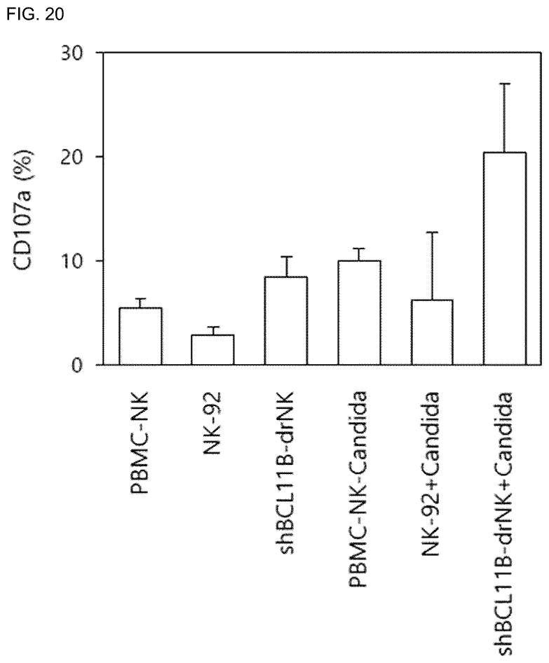

In an exemplary embodiment of the present invention, the antifungal effect of drNK cells against Candida Albicans, a type of Candida fungus, was examined through the expressed CD107a⁺ cell frequency after co-culture, and as a result, it was found that drNK exhibited higher antifungal efficacy compared to NK-92 and PBMC-NK cells (FIG. 20).

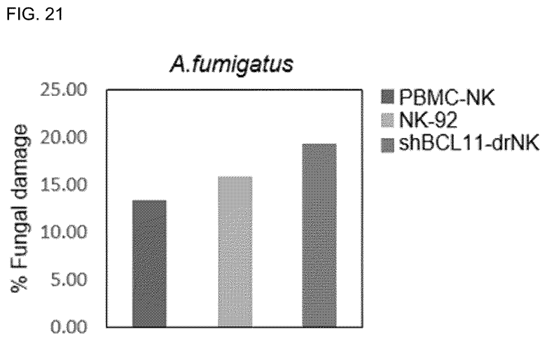

In another exemplary embodiment of the present invention, it was found that both shBCL11B-drNK cells and positive controls (PBMC-NK cells and NK-92 cells) exhibit antifungal activity against Aspergillus fumigatus and particularly shBCL11B-drNK cells exhibited superior antifungal activity (FIG. 21).

The cell therapeutic composition, the content of drNK cells and/or CAR-drNK cells in the composition, and the dosage of the cell therapeutic composition are as described above.

Still another aspect of the present invention provides a pharmaceutical composition for the treatment or prevention of infectious diseases and/or inflammatory diseases, containing the cells prepared by the method of the present invention as an active ingredient.

The terms used here are as described above.

The pharmaceutical composition, the content of drNK cells and/or CAR-drNK cells in the composition, and the dosage of the pharmaceutical composition are as described above.

Still another aspect of the present invention provides a method for treating infectious diseases and/or inflammatory diseases, the method including administering the cell therapeutic composition or pharmaceutical composition to a subject.

The terms used here are as described above.

Still another aspect of the present invention provides a medium kit for direct cell-fate conversion induction for the preparation of drNK cells or CAR-drNK cells, the medium kit including: a) a first container containing i) BCL11B shRNA, ii) BCL11B siRNA, or iii) CRISPR/Cas9-gRNA-BCL11B; b) a second container containing the first medium of the present invention; and c) a third container containing the second medium of the present invention.

The terms used here are as described above.

The kit of the present invention refers to a tool that may be used as a direct reprogramming medium for the preparation of drNK cells or CAR-drNK cells by containing the first container containing i) BCL11B shRNA, ii) BCL11B siRNA, or iii) CRISPR/Cas9-gRNA-BCL11B, the second container containing the first medium of the present invention, and the third container containing the second medium of the present invention. The type of kit is not particularly limited, and kits in the form commonly used in the art may be used.

The kit of the present invention may be packaged in a form in which the i) BCL11B shRNA, ii) BCL11B siRNA, or iii) CRISPR/Cas9-gRNA-BCL11B; the first medium; and the second medium are contained in individual containers, or in a form in which these are contained in one container divided into one or more compartments, and the i) BCL11B shRNA, ii) BCL11B siRNA, or iii) CRISPR/Cas9-gRNA-BCL11B; the first medium; and the second medium may each be packaged in unit dose form by a single administration dosage.

The i) BCL11B shRNA, ii) BCL11B siRNA, or iii) CRISPR/Cas9-gRNA-BCL11B; the first medium; and the second medium in the kit may be administered sequentially at an appropriate time according to the experimental plan by those skilled in the art.

The kit of the present invention may further include instructions for use that describe the added amount, addition method, and addition frequency of each of the i) BCL11B shRNA, ii) BCL11B siRNA, or iii) CRISPR/Cas9-gRNA-BCL11B; the first medium; and the second medium.

### DETAILED DESCRIPTION OF THE INVENTION

Hereinafter, the configuration and effects of the present invention will be described in more detail with reference to Examples. However, these Examples are for illustrative purposes only, and the scope of the present invention is not limited by these Examples.

### Example 1: Preparation of shBCL11B-drNK and Verification of Expression of NK (Natural Killer) Cell-Specific Markers

1-1. Preparation of shBCL11B-drNK

In order to induce direct cell-fate conversion through inhibition of BCL11B (B-cell lymphoma/leukemia 11B) gene expression in cells, short hairpin RNA (shRNA) against BCL11B (shBCL11B) or siRNA was introduced as a reprogramming factor that induces cell-fate conversion into somatic cells.

Specifically, peripheral blood mononuclear cells (PBMC) as a somatic cell were treated with lentiviruses each expressing 7 types of shBCL11Bs (shBCL11B#1, shBCL11B#2, shBCL11B#3, shBCL11B#4 shBCL11B#5, shBCL11B#6, and shBCL11B#7) or four types of siRNAs (siBCL11B-A, siBCL11B-B, siBCL11B-C, and siBCL11B-D) as reprogramming factors to introduce shBCL11B or siRNA (FIG. 4A), whereby shBCL11B-drNK cells were prepared.

In order to introduce 7 types of lentiviruses expressing shBCL11B into PBMC cells, 2×10⁵ PBMC cells were seeded in 48-well plates, treated with the lentiviruses at an MOI of 3 and 4 μg/ml polybrene or 6 μM Bx795, and cultured in RPMI medium for 16 hours, and then the medium was replaced with fresh medium to transform the PBMC cells.

In order to introduce four types of siRNAs into PBMC cells, 2×10⁵ PBMC cells were seeded in 48-well plates and treated with 100 nM siRNA using Lipofectamine RNAiMAX (Thermo Fisher Scientific Inc.) according to the manufacturer's instructions to transform the PBMC cells.

The next day, 2×10⁵ transformed cells were seeded in 48-well plates, cultured for 6 days in the first medium (StemSpan SFEM II containing 3 μM CHIR99021 (CT99021), 10% FBS (fetal bovine serum), 1% penicillin/streptomycin, 20 ng/ml human SCF, 20 ng/ml human FMS-like tyrosine kinase ligand (FLT3L), 20 ng/ml human IL-7, 200 IU/ml human IL-2, and 20 ng/ml human IL-15) containing a GSK3β (glycogen synthase kinase 3β) inhibitor, and then cultured for 11 days in the second medium (StemSpan SFEM II containing 10% FBS, 1% penicillin/streptomycin, 20 ng/ml human SCF, 20 ng/ml human FLT3L, 200 IU/ml human IL-2, 20 ng/ml human IL-7, 20 ng/ml human IL-15, and 2 μM StemRegenin 1 (SR1)) containing an AHR (aryl hydrocarbon receptor) inhibitor.

A schematic diagram of the method for preparing shBCL11B-drNK by direct cell-fate conversion of somatic cells through the introduction of shBCL11B according to the description above is as illustrated in FIGS. 1A and 4B.

### 1-2. Examination of NK Cell Preparation Yield

In order to examine the production and yield of the shBCL11B-drNK cells of Example 1-1, the cells were stained with CD56 antibody and CD3 antibody, and the ratio of NK cell populations (CD56⁺ and CD3⁻) was analyzed using a flow cytometer.

As a result, NK cells (CD56⁺CD3⁻) were present at a ratio of 2.1% (No-treated) and 0.2% (sh-Control) in the controls, respectively. However, ratios of 76.1%, 90.4%, 85.1%, 72.6%, 91.7%, 77.9%, 90.3%, 57.1%, 48.7%, 38.2%, or 42.3% were observed in shBCL11B-drNK cells prepared by introducing or transforming with shBCL11B#1, shBCL11B#2, shBCL11B#3, shBCL11B#4 shBCL11B#5, shBCL11B#6, shBCL11B#7, siBCL11B-A, siBCL11B-B, siBCL11B-C, or siBCL11B-D, and it was found that NK cells were prepared at high efficiency when shBCL11B or siRNA was introduced into cells (FIG. 4C).

### 1-3. Verification of Expression Characteristics of NK-Specific Markers

In order to verify the expression characteristics of NK-specific markers of the shBCL11B-drNK cells of Example 1-1, only NK cells were recovered by MACS (magnetic activated cell sorting) using the NK isolation Kit (Miltenyl Biotec), and the expression patterns of activating receptors (CD16, CD69, NKG2D, NKp30, NKp44, NKp46, and DNAM-1) or inhibitory receptors (KIR2DL1, KIR2DL2, and KIR3DL1) related to NK cells were analyzed using a flow cytometer.

As a result, it was found that activating receptors such as CD16, CD69, NKG2D, NKp30, NKp44, NKp46, and DNAM-1 were expressed at a higher frequency than inhibitory receptors such as KIR2DL1, KIR2DL2, and KIR3DL1 in shBCL11B-drNK cells (FIG. 6).

Through this, it has been found that somatic cells are converted into NK cells (shBCL11B-drNK) through direct cell-fate conversion of somatic cells using shBCL11B.

### Example 2: Preparation of CAR-shBCL11B-drNK and Verification of Expression Characteristics of NK-Specific Markers

### 2-1. Preparation of CAR-shBCL11B-drNK

GAR-shBCL11B-drNK cells expressing a CAR (chimeric antigen receptor) gene were constructed by simultaneously introducing shBCL11B and CAR genes into cells.

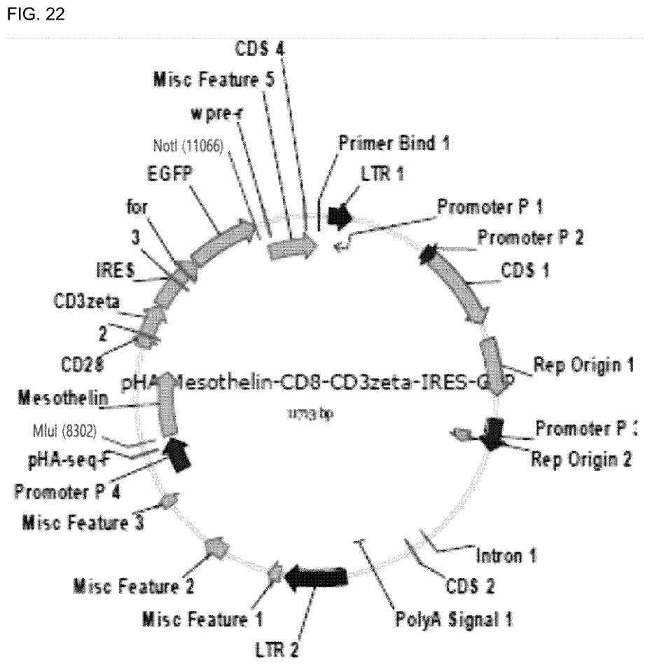

Specifically, a double cistron lentiviral vector gene encoding MSLN (mesothelin)-specific MSLN-CAR was constructed (FIG. 22). The MSLN-CAR gene was configured to contain CD8 leader (SEQ ID NO: 14), MSLN (Mesothelin) scFv (SEQ ID NO: 16), CD8 hinge (SEQ ID NO: 18), CD8 transmembrane domain (SEQ ID NO: 19), CD28 intracellular domain (SEQ ID NO: 21), CD3ζ (SEQ ID NO: 22), IRES (SEQ ID NO: 23), and GFP (SEQ ID NO: 24) (FIG. 3A).

PBMC cells were transformed with lentivirus expressing shBCL11B#2 on day 0, and with lentivirus expressing an MSLN-CAR gene on day 0, day 6, day 12, and day 18. During this period, cell culture from day 0 to day 18 was performed in the same manner as in Example 1-1, that is, the cells were cultured in the first medium from day 0 to day 7 and in the second medium thereafter to prepare ShBCL11B-drNK (MSLN-shBCL11B-drNK) expressing an MSLN-CAR gene (FIG. 11A).

A schematic diagram of the method for preparing CAR-shBCL11B-drNK by direct cell-fate conversion of somatic cells through the introduction of shBCL11B and CAR genes according to the description above is as illustrated in FIGS. 1B and 11A.

### 2-2. Examination of NK Cell Preparation Yield

In order to examine the production and yield of the MSLN-shBCL11B-drNK cells of Example 2-1, the cells were stained with CD56 antibody and MSLN-CAR antigen, and the ratio of NK cell populations (CD56⁺ and CD3⁻) was analyzed using a flow cytometer.

As a result, it was found that CD56⁺MSLN⁺(MSLN-shBCL11B-drNK) cells were prepared at the highest efficiency in the cell population transformed on day 6 among cells transformed on day 0 (4.9%), day 6 (13.1%), day 12 (9.9%), and day 18 (8.8%) (FIG. 11B).

### 2-3. Verification of Expression Characteristics of NK-Specific Markers

In order to verify the expression characteristics of NK-specific markers of the MSLN-shBCL11B-drNK cells of Example 2-1, the expression patterns of various activating and inhibitory receptors related to natural killer cells of MSLN-shBCL11B-drNK cells were analyzed using a flow cytometer in the same manner as in Example 1-3.

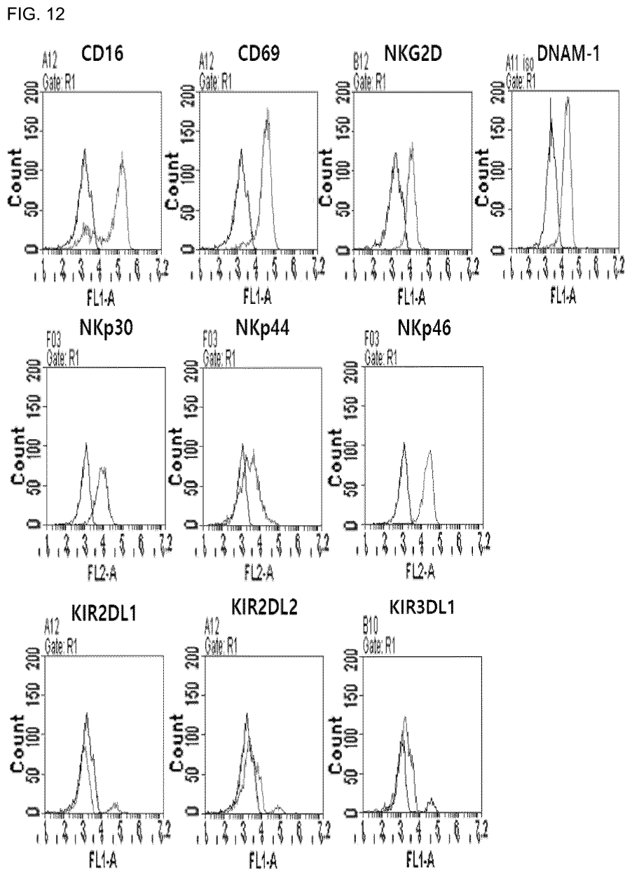

As a result, it was found that in the prepared MSLN-shBCL11B-drNK, activating receptors such as CD16, CD69, NKG2D, NKp30, NKp44, NKp46, and DNAM-1 were expressed at a higher frequency than inhibitory receptors such as KIR2DL1, KIR2DL2 and KIR3DL1 (FIG. 12), showing a similar tendency to that in shBCL11B-drNK.

### Example 3: Preparation of gBCL11B-drNK Using CRISPR (Clustered Regularly Interspaced Short Palindromic Repeats)/Cas9 and Verification of Expression Characteristics of NK-Specific Markers

3-1. Preparation of gBCL11B-drNK

In order to induce direct cell-fate conversion through inhibition of BCL11B (B-cell lymphoma/leukemia 11B) gene expression in cells, a commercial lentiviral vector (Applied Biological Material, CAT #: K0013105) into which the genetic scissors CRISPR/Cas9 and sgRNA (single-guide RNA) were introduced as a cell-fate conversion inducing factor was used.

In the CRISPR/Cas9 system of the present Example, the sgRNA included sgRNA-A, sgRNA-B, or sgRNA-C contained in the commercial lentiviral vector set.

Specifically, in order to transform PBMC cells with a reprogramming factor, a lentivirus at an MOI of 3 expressing CRISPR/Cas9 vector containing sgRNA-A, sgRNA-B, or sgRNA-C, PBMC cells, and polybrene (4 μg/ml) were cultured together for 16 hours and then the medium was replaced with fresh medium to transform the PBMC cells. Thereafter, cell culture from day 0 to day 18 was performed in the same manner as in Example 1-1, that is, the cells were cultured in the first medium from day 0 to day 7 and in the second medium thereafter to prepare direct cell-fate conversion induced drNK cells (gBCL11B-drNK) (FIG. 13A).

A schematic diagram of the method for preparing gBCL11B-drNK by direct reprogramming of somatic cells through the introduction of the genetic scissors CRISPR/Cas9 is as illustrated in FIGS. 2A and 13A.

### 3-2. Examination of NK Cell Preparation Yield

In order to examine the production and yield of the gBGL11B-drNK cells of Example 3-1, the cells were stained with CD56 antibody and CD3 antibody, and the ratio of NK cell populations (CD56⁺ and CD3⁻) was analyzed using a flow cytometer.

As a result, it was found that CD56⁺CD3⁻(gBCL11B-drNK) cells were prepared at an efficiency of 83.9% (sgRNA-A), 72.2% (sgRNA-B), and 72.0% (sgRNA-C) in the Cas9 and sgRNA treated groups, respectively, compared to the No-treated group (6.0%) (FIG. 13B)

### 3-3. Verification of Expression Characteristics of NK-Specific Markers

In order to verify the expression characteristics of NK-specific markers of the gBCL11B-drNK cells of Example 3-1, the expression patterns of various activating and inhibitory receptors related to natural killer cells of the gBCL11B-drNK cells were analyzed using a flow cytometer in the same manner as in Example 1-3.

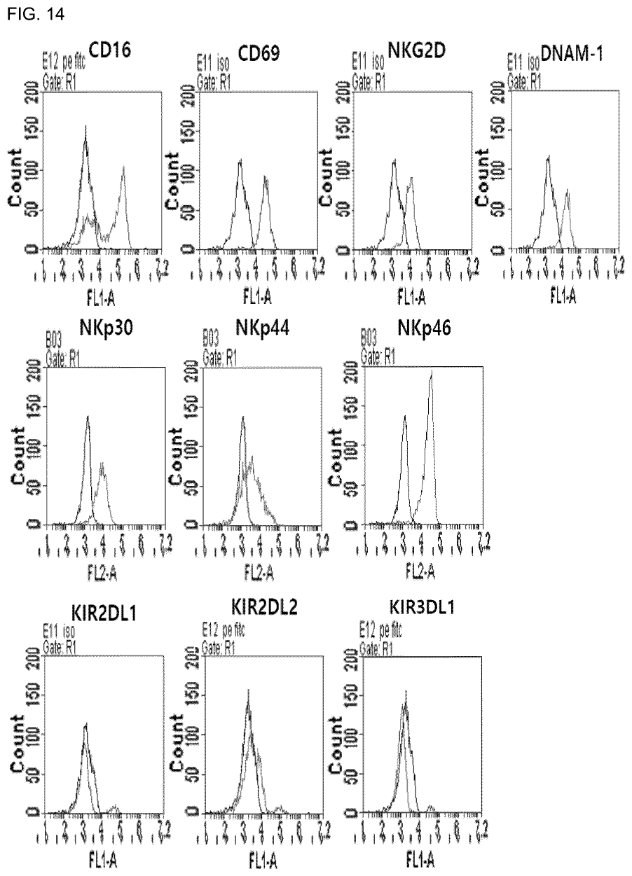

As a result, it was found that in the prepared gBCL11B-drNK cells, activating receptors such as CD16, CD69, NKG2D, NKp30, NKp44, NKp46, and DNAM-1 were expressed at a higher frequency than inhibitory receptors such as KIR2DL1, KIR2DL2 and KIR3DL1 (FIG. 14), showing a similar tendency to that in shBCL11B-drNK.

Accordingly, it has been found that drNK is prepared through the introduction of Cas9/sgRNA as well as through the introduction of shBCL11B.

### Example 4: Preparation of CAR-gBCL11B-drNK and Verification of Expression Characteristics of NK-Specific Markers

### 4-1. Preparation of CAR-gBCL11B-drNK

CAR-gBCL11B-drNK cells expressing a CAR gene were constructed by simultaneously introducing Cas9, sgRNA, and CAR gene into cells.

In the CRISPR/Cas9 system of the present Example, the sgRNA included sgRNA #1 (forward) and sgRNA #2 (reverse) in Table 1 below targeting Exon1 (BCL11B-ex1) derived from the genomic sequence (NC_000014.9) containing BCL11B.

TABLE 1

| SEQ ID NO: | Sequence name | Sequence (5′→3′) |
|---|---|---|
| 12 | sgRNA#1 | GGCAATGTCCCGCCGCAAACAGG |
| | (forward) | (SEQ ID NO: 12) |
| 13 | sgRNA#2 | GCGGGTTGCCCTGTTTGCGGCGG |
| | (reverse) | (SEQ ID NO: 13) |

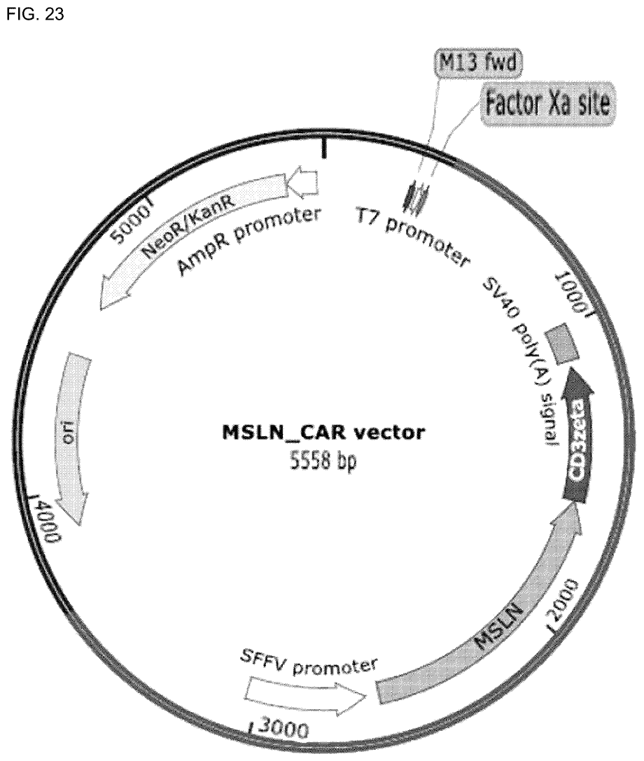

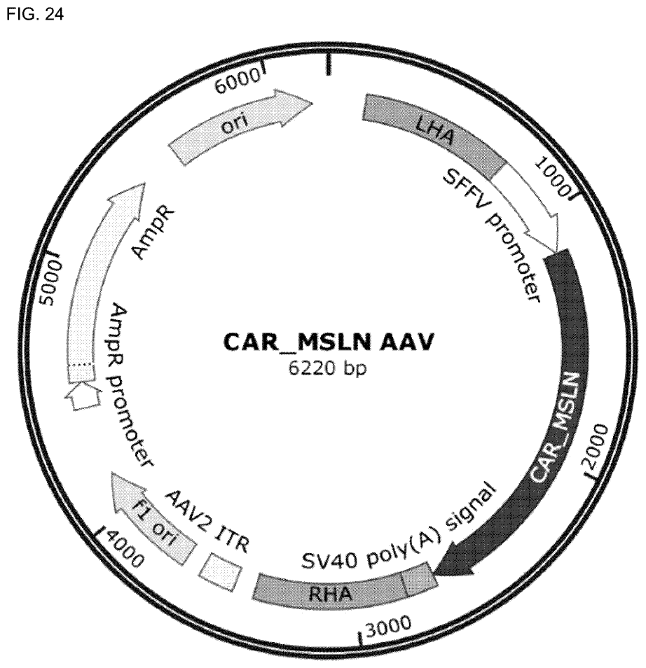

Specifically, in order to construct a donor plasmid in which the CAR coding sequence could be inserted and knocked-in (KI) into the cut site at the same time as BCL11B knock-out (KO), centering on BCL11B-exon1, the right homology arm (RHA) 600 bp (99270561-99271160 base sequence in NC_000014.9) (SEQ ID NO: 25) and the left homology arm (LHA) 600 bp (99271219-99271818 base sequence in NC_000014.9) (SEQ ID NO: 26) were selected in the intron portion, respectively, and a CAR plasmid containing SEFV promoter (SEQ ID NO: 27) and polyA signal (SEQ ID NO: 28) between LHA and RHA was constructed (FIG. 23). Thereafter, the 2707-5740 bp region of the MSLN-CAR plasmid was inserted into the site where the 150-787 bp region was removed from pJEP300-pAAV-CMV-MCS2-pA to construct CAR-AAV (FIGS. 3B and 24).

PBMC cells were cultured in RPMI medium for 1 day, and then treated with CAR-AAV (1 MOI) encoding MSLN-CAR one day before the start of the experiment (day −1) for transformation. On day 0, 24 hours later, 1×10⁶ PBMCs treated with AAV were further transformed with a plasmid vector containing sgRNA #1 and sgRNA #2 by electroporation. The transformed cells were cultured in RPMI medium for 1 day, 5×10⁵ cells were then cultured in the first medium until day 6 and in the second medium thereafter in the same manner as in Example 1-1 to prepare direct cell-fate conversion induced drNK cells (MSLN-gBCL11B-drNK) expressing a CAR (MSLN-CAR) gene (FIGS. 15A and 15B).

A schematic diagram of the method for preparing CAR-gBCL11B-drNK by direct reprogramming of somatic cells through the introduction of Cas9, sgRNA, and CAR gene is as illustrated in FIGS. 2B and 15A.

### 4-2. Examination of NK Cell Preparation Yield

In order to examine the production and yield of the MSLN-gBGL11B-drNK cells of Example 4-1, the cells were stained with CD56 antibody, CD3 antibody, and MSLN-CAR antigen, and then the ratio of NK cell populations (CD56⁺, CD3⁻, and MSLN-CAR⁺) was analyzed using a flow cytometer.

As a result, it was found that the CD56⁺MSLN⁺ cell population was prepared at an efficiency of 17.3% compared to the CD56⁺CD3⁻ cell population (22.8%) (FIG. 15C).

Next, the insertion of CAR gene into the drNK cell genome was examined through PCR analysis.

Specifically, PCR was performed on genomic DNA using a primer for the pre-LHA sequence (SEQ ID NO: 29) and a primer for the SFFV promoter (SEQ ID NO: 30). In order to extract genomic DNA (gDNA) from each cell, total DNA was extracted using the DNeasy Blood & Tissue kit (QIAGEN, cat. no. 69504) according to the manufacturer's protocol. For each sample, 200 ng of gDNA, 2× Premix (EmeraldAmp GT PCR master mix, TAKARA, cat. no. RR310A), and target primers were mixed to prepare a total of 20 μl of reaction solution. This was subjected to PCR by denaturation at 95° C. for 10 minutes, 40 cycles of annealing at 95° C. for 30 seconds, at 57° C. for 40 seconds, and at 72° C. for 1 minute, and elongation at 72° C. for 5 minutes. The reacted PCR product was subjected to electrophoresis on a 1% agarose gel.

The primer sequences used here are as listed in Table 1 below.

TABLE 1

| SEQ ID NO: | Sequence name | Sequence (5′→3′) |
|---|---|---|
| 29 | LHA primer | ACC GAA CCG GGG CAG TTT TA (SEQ ID NO: 29) |
| 30 | SFFV promoter primer | TTT TCA TGT ACC CGC CCT TGA T (SEQ ID NO: 30) |

As a result, it was found that the CAR gene was inserted into the drNK cell genome (CAR knock-in, CAR-KI) (FIG. 15D).

Accordingly, it has been found that in a case where GAR-gBGL11B-drNK cells are prepared by simultaneously introducing Cas9, sgRNA, and CAR gene, the preparation yield of drNK cells is lower compared to CAR-shBCL11B-drNK, but the CAR gene can be specifically inserted only into the genome where BCL11B is located, and the possibility of genetic modification by lentivirus can be minimized.

### Example 5: Effect of Media Components on shBCL11B-drNK Preparation Yield

5-1. shBCL11B-drNK Preparation Yield Depending on Composition of First Medium

shRNA #2 against BCL11B was introduced into PBMCs in the same manner as Example 1-1, and then the PBMCs were cultured for 6 days in the first medium (positive control) or media each lacking one of 200 IU/ml human IL-2, 20 ng/ml human IL-15 or 3 μM CHIR99021 among the components constituting the first medium or a medium further containing 6 μM BX795 in addition to the components constituting the first medium, and then further cultured for 12 days in the second medium.

In order to examine whether NK cells were prepared through the culture, the cells were stained with CD56 antibody and CD3 antibody, and then the ratio of drNK cell populations (CD56⁺ and CD3⁻) was analyzed using a flow cytometer.

As a result, it was found that CD56⁺CD3⁻(shBCL11B-drNK) cells were prepared at an efficiency of 43% in the cell population cultured in the medium lacking IL-2, 75% in the cell population cultured in the medium lacking IL-15, 92% in the cell population cultured in the medium lacking CHIR99021, and 113% in the cell population cultured in the medium further containing BX795 based on the efficiency (100%) in the positive control cultured in the first medium (FIG. 5A).

5-2. shBCL11B-drNK Preparation Yield Depending on Composition of Second Medium

shRNA #2 against BCL11B was introduced into PBMCs in the same manner as in Example 1-1, and then the PBMCs were cultured for 6 days in the first medium and further cultured for 12 days in the second medium (positive control) or media, each lacking one of 200 IU/ml human IL-2, 20 ng/ml human IL-7, 20 ng/ml human IL-15, 20 ng/ml human FLT3L, 20 ng/ml human SCF, or 2 μM SR1.

In order to examine whether NK cells were prepared through the culture, the cells were stained with CD56 antibody and CD3 antibody, and then the ratio of drNK cell populations (CD56⁺ and CD3⁻) was analyzed using a flow cytometer.

As a result, it was found that the CD56⁺CD3⁻(shBCL11B-drNK) cells were prepared at an efficiency of 56% in the cell population cultured in the medium lacking IL-2, 69% in the cell population cultured in the medium lacking IL-15, 82% in the cell population cultured in the medium lacking IL-7, 81% in the cell population cultured in the medium lacking SCF, 38% in the cell population cultured in the medium lacking FLT3L, and 57% in the cell population cultured in the medium lacking SR1 based on the efficiency (100%) in the positive control cultured in the second medium (FIG. 5B).

### Example 6: Verification of Efficacy of shBCL11B-drNK Cells

6-1. Measurement of Cancer Cell-Killing Ability of shBCL11B-drNK Cells

In order to measure the cancer cell-killing ability of the shBCL11B-drNK (shRNA #2) cells of Example 1-1, a cancer cell-killing ability measurement using calcein-AM was performed.

Specifically, Raji (human B-lymphoma), HCT116 (human colorectal carcinoma cell line), SNU-817 (B lymphoma cell line), HepG2 (hepatocellular carcinoma cell line), NCIH460 (human non-small cell lung cancer), Mia-paca-2 (hypotriploid human pancreatic cancer cell line), U373MG (human glioblastoma astrocytoma), SW620 (human colon carcinoma cell line), SK-OV-3 (human ovarian cancer cell line), MCF7 (breast cancer cell line), PC-3 (human prostate cancer cell line), SK-MEL-3 (human melanoma cell lines), A-673 (human rhabdomyosarcoma), Caki-1 (human clear cell renal cell carcinoma), SNU-790 (human thyroid papillary carcinoma cell line), MG-63 (human osteosarcoma cell line), BeWo (human placental choriocarcinoma), KATO III (human gastric carcinoma) and 253j (human bladder cancer cell line) as cancer cells were diluted to 1×10⁵ cells/ml in DMEM medium containing 10% FBS, then calcein-AM was added to have a final concentration of 25 μM, the cells were cultured at 37° C. for 1 hour, and then washed with DMEM medium to prepare calcein-labeled target cancer cells.

The culture solution of the shBCL11B-drNK (shRNA #2) cells of Example 1-1 was diluted to have a density of 0.25×10⁵ cells/ml, 1×10⁵ cells/ml, and 2.5×10⁵ cells/ml and then dispensed into 96-well plates by 100 ml. The calcein-labeled target cancer cells (1×10⁵ cells/ml) prepared was added into the 96-well plates by 100 μl/well, then centrifugation was performed at 400 g for 1 minute, and co-culture was performed for 4 hours in an incubator at 37° C. and 5% CO2. The supernatant was taken from each well by 100 μl and subjected to the measurement using a fluorescence plate reader (485 nm/535 nm). The cancer cell-killing ability (%) was calculated according to the equation below.

$$Cancer cell - killing ability ( \% ) = \{ ( measured value - minimum value ) / ( maximum value - minimum value ) \} \times 100$$

In the equation, the minimum value is the measured value for a well in which only calcein-labeled target cancer cells are present, and the maximum value is the measured value for a well in which the calcein-labeled target cancer cells were completely lysed by adding 1.0% TritonX-100 to the cells.

As a result, it was found that shBCL11B-drNK cells had the ability to kill various types of cancer cells and that the cancer cell-killing ability increased as the number of shBCL11B-drNK cells with respect to the number of cancer cells increased (FIG. 7).

6-2. Comparison of Cancer Cell-Killing Ability Between shBCL11B-drNK Cells and Existing Human Natural Killer (NK) Cells

The cancer cell-killing ability against K562 (human myelogenous leukemia cell line) between shBCL11B-drNK cells of Example 6-1 and NK-92 cells (ATCC), an existing human natural killer cell, (positive control) was compared in the same manner as in Example 6-1.

As a result, it was found that shBCL11B-drNK cells were superior to NK-92, a control, in the cancer cell-killing ability by about 5.2 to 5.5 times (FIG. 8).

6-3. Comparison of Frequencies of CD107a⁺ Cells and Interferon Gamma (IFN-Gamma) Expressing Cells on Cancer Cell Sensitization Between shBCL11B-drNK Cells and Natural Killer Cells Derived from Human Peripheral Blood Cells

In a case where NK cells are co-cultured with cancer cells, CD107a⁺ cells having cancer cell lytic ability are expressed, and it was examined whether CD107a⁺ cells were expressed when shBCL11B-drNK cells were co-cultured with cancer cells.

The shBCL11B-drNK cells of Example 6-1 or PBMC-NK cells, natural killer cells derived from human peripheral blood cells, (positive control) were co-cultured with cancer cells (HCT116, HepG2, and Mia-paca-2) in an incubator at 37° C. and 5% CO2 for 4 hours, and the expressed CD107a⁺ cells with cancer cell lytic ability were quantitatively analyzed.

PBMC-NK cells were isolated from PBMC cells using an NK isolation kit. The PBMC-NK cells were isolated from PBMC cells and then cultured in NK medium (RPM11640 containing 1% penicillin/streptomycin, 200 IU/ml human IL-2, and 20 ng/ml human IL-15) for 2 days before use.

As a result, in a case where shBCL11B-drNK cells or PBMC-NK cells were co-cultured with cancer cells, the frequency (%) of CD107a⁺ cells increased in both cases compared to the control (No target) not undergone co-culture (FIG. 9A). The frequency of CD107a⁺ cells further increased in a case where shBCL11B-drNK cells and cancer cells were co-cultured compared to a case where PBMC-NK cells and cancer cells were co-cultured.

Next, the shBCL11B-drNK cells of Example 6-1 or PBMC-NK cells (positive control) were co-cultured with cancer cells (HCT116, HepG2, and Mia-paca-2) in the same manner as above, and then the expressed IFN-gamma⁺ cells were quantitatively analyzed.

As a result, it was found that in a case where shBCL11B-drNK cells or PBMC-NK cells were co-cultured with cancer cells, the frequency (%) of IFN-gamma⁺ cells increased in both cases compared to the control (No target) not undergone co-culture (FIG. 9B). The frequency of IFN-gamma⁺ cells significantly increased in a case where shBCL11B-drNK cells and HepG2 were co-cultured compared to a case where PBMC-NK cells and HepG2 were co-cultured.

6-4. Comparison of In Vivo Cancer Cell-Killing Ability Between shBCL11B-drNK Cells and Natural Killer Cells Derived from Human Peripheral Blood Cells

Each luciferase-expressing cancer cell (PC-3 or SK-OV-3) was subcutaneously injected into the back of an 8-week-old nude mouse (Balb/c-nude mouse, average weight 20 to 25 g) by 200 μl at 1×10⁷ cells/ml to prepare a mouse prostate cancer model or a mouse ovarian cancer model.

The next day, 200 μl of phosphate-buffered saline (PBS) as a negative control, PBMC-NK cells as a positive control or shBCL11B-drNK cells of Example 6-1 were injected into the tail vein by the same volume (1×10⁷ cells/150 μl), and then the tumor size was measured at 7-day intervals until day 21. The PBMC-NK cells were isolated from PBMC cells and then cultured in NK medium for 2 days before use.

As a result, the tumor size was significantly reduced on day 21 after injection of each cell or PBS into mice bearing PC-3 tumor in the PBMC-NK cell administered group (7.31×10⁹ radiance) and shBCL11B-drNK cell administered group (6.07×10⁹ radiance) compared to the tumor size of the control (4.66×10¹⁰ radiance), and it was found that shBCL11B-drNK cells exhibited a superior anticancer effect compared to PBMC-NK cells (FIG. 10A).

The tumor size was significantly reduced on day 21 after injection of each cell or PBS into mice bearing SK-OV-3 tumor in the PBMC-NK cell administered group (1.14×10¹⁰ radiance) and shBCL11B-drNK cell administered group (5.92×10⁹ radiance) compared to the tumor size of the control (3.12×10¹⁰ radiance), and it was found that shBCL11B-drNK cells exhibited a superior anticancer effect compared to PBMC-NK cells (FIG. 10B).

### Example 7: Verification of Anticancer Efficacy of drNK Cells or CAR-drNK Cells

In order to verify the cancer cell-killing ability of the shBCL11B-drNK (shRNA #2) cells prepared in Example 1-1, the MSLN-shBCL11B-drNK (shRNA #2) cells prepared in Example 2-1, the gBCL11B-drNK (sgRNA-A) cells prepared in Example 3-1, and the MSLN-gBCL11B-drNK (sgRNA #1 and sgRNA #2) cells prepared in Example 4-1, the cells were co-cultured with K562, which did not express MSLN, and with PC-3 and Mia-paca-2, which expressed MSLN, as cancer cells in the same manner as in Example 6-1, and the cancer cell-killing ability thereof was analyzed.

As a result, the four types of drNK cells exhibited similar cancer cell-killing ability against K562, which did not express MSLN (FIG. 16A).

However, against Mia-paca-2, which expressed MSLN, MSLN-shBCL11B-drNK cells (21.9%) and MSLN-gBCL11B-drNK cells (20.8%), CAR(MSLN)-drNK cells, exhibited a higher cancer cell-killing ability compared to shBCL11B-drNK cells (3.9%) and gBCL11B-drNK cells (4.8%), non-CAR-drNK cells, when the number of drNK cells with respect to the number of cancer cells was 1:1, and it was found that the cancer cell-killing ability increased as the number of CAR-drNK cells with respect to the number of cancer cells increased (FIG. 16A).

Against PC-3, which expressed MSLN, MSLN-shBCL11B-drNK cells (41.4%) and MSLN-gBCL11B-drNK cells (44.7%), CAR(MSLN)-drNK cells, exhibited a higher cancer cell-killing ability compared to shBCL11B-drNK cells (25.1%) and gBCL11B-drNK cells (15.0%), non-CAR-drNK cells, when the number of drNK cells with respect to the number of cancer cells was 1:1, and it was found that the cancer cell-killing ability increased as the number of CAR-drNK cells with respect to the number of cancer cells increased (FIG. 16A).

Next, in order to verify the cancer cell killing potential of the shBCL11B-drNK cells, MSLN-shBCL11B-drNK cells, gBCL11B-drNK cells, and MSLN-gBCL11B-drNK cells, CD107a⁺ cells expressed by co-culture were quantitatively analyzed in the same manner as in Example 6-3.

As a result, it was found that against K562, which did not express MSLN, the frequency (%) of CD107a⁺ cells decreased in MSLN-shBCL11B-drNK and MSLN-gBCL11B-drNK cells compared to shBCL11B-drNK and gBCL11B-drNK, but against PC-3, which expressed MSLN, the frequency (%) of CD107a⁺ cells increased in MSLN-shBCL11B-drNK (32.0%) and MSLN-gBCL11B-drNK (37.2%) cells, CAR(MSLN)-drNK cells, compared to shBCL11B-drNK (17.6%) and gBCL11B-drNK (19.4%), non-CAR-drNK cells (FIG. 16B). It was found that the CD107a⁺ cell frequency against Mia-paca-2, which expressed MSLN, was similar to that against PC-3.

### Example 8: Verification of Antiviral Efficacy of drNK Cells

In order to examine the antiviral effect of the shBCL11B-drNK (shRNA #2) cells of Example 1-1, the cell-killing ability against Ramos (human B-lymphoma), which was not infected with a virus, and Raji, which was infected with Epstein-Barr virus (EBV) was measured in the same manner as in Example 6-1. PBMC-NK cells or NK-92 cells were used as a positive control.

As a result, the cell-killing ability of both the shBCL11B-drNK cells (BCL11B-drNK) and the positive controls (PBMC-NK cells and NK-92 cells) against EBV-infected Raji was higher than that against uninfected Ramos, and the shBCL11B-drNK cells exhibited a higher cell-killing ability against both uninfected Ramos and EBV-infected Raji compared to the positive controls (PBMC-NK cells and NK-92 cells) (FIG. 17A).

Next, EBV-infected Raji and uninfected Ramos were co-cultured with drNK cells and the frequency of CD107a⁺ cells was determined.

Specifically, 1×10⁶ cells/ml of EBV-infected Raji or uninfected Ramos and 1×10⁶ cells/ml of shBCL11B-drNK cells, PBMC-NK cells as a positive control, or NK-92 cells as a positive control were dispensed into a 6-well plate by 1 ml each, then centrifugation was performed at 400 g for 1 minute, co-culture was performed for 4 hours in an incubator at 37° C. and 5% CO2, then the cells were washed and recovered using a centrifuge, and FACS (fluorescence-activated cell sorting) analysis was performed to determine the frequency of CD107a⁺ cells responding to the infected cells.

In order to determine the frequency of CD107a⁺ cells, 1×10⁶ cells/ml of shBCL11B-drNK cells, PBMC-NK cells as a positive control or NK-92 cells as a positive control were put into FACS buffer containing fluorescent labeled antibodies against CD56 and CD107a and reacted at room temperature for 20 minutes, and then the cells were washed and recovered using a centrifuge and subjected to FACS analysis.

As a result, the frequency (%) of CD107a⁺ cells further increased when both the shBCL11B-drNK cells (BCL11B-drNK) and the positive controls (PBMC-NK cells and NK-92 cells) were co-cultured with EBV-infected Raji rather than Ramos, and the frequency of CD107a⁺ cells significantly increased in the case of shBCL11B-drNK cells compared to the case of positive controls (PBMC-NK cells and NK-92 cells) (FIG. 17B).

Next, EBV-infected Raji and uninfected Ramos were co-cultured with drNK cells and the expression level of LMP-1 (latent membrane protein 1), an EBV-specific gene was examined.

Specifically, Raji was transformed into GFP-Raji through treatment with a lentivirus at an MOI of 5 obtained from a GFP-expressing lentiviral vector (control vector, ORIGENE, CAT #: TL306424 in CTIP2 (BCL11B) Human shRNA Plasmid Kit (Locus ID 64919)) and 8 μg/ml polybrene, cultured in RPMI medium for 16 hours and then the medium was replaced with fresh medium.

Into a 6-well plate, 1×10⁶ cells/ml of the prepared GFP-Raji and 1×10⁶ cells/ml of shBCL11B-drNK cells were dispensed by 1 ml each, pipetted, and co-cultured for 24 hours in an incubator at 37° C. and 5% C02, and the reaction product was recovered by centrifugation. PBMC-NK cells or NK-92 cells were used as a positive control. Total RNA was extracted from the cell reaction product using the RNeasy Mini kit (Qiagen) and reverse transcribed using the SuperScript VILOTM cDNA Synthesis Kit (Thermo Fisher Scientific Inc.) according to the manufacturer's instructions. qRT-PCR was performed with SYBR Green, and the expression level of LMP-1 was analyzed using the 7500 Fast real-time PCR system (Applied Biosystems).

The primer sequences used here are as listed in Table 2 below.

TABLE 2

| SEQ ID NO: | Sequence name | Sequence (5′→3′) |
|---|---|---|
| 31 | LMP-1 Forward | TCCTCCTGTTTCTGGCGATT (SEQ ID NO: 31) |
| 32 | LMP-1 Reverse | GGAGTCATCGTGGTGGTGTTC (SEQ ID NO: 32) |
| 33 | GFP Forward | ATGGTGAGCAAGGGCGAGGAG (SEQ ID NO: 33) |
| 34 | GFP Reverse | CGGTGGTGCAGATGAACTTCAGG (SEQ ID NO: 34) |

As a result, it was found that the expression level of LMP-1 further decreased compared to GFP in both a case where the shBCL11B-drNK cells were co-cultured with GFP-Raji cells and a case where the positive controls (PBMC-NK cells and NK-92 cells) were co-cultured with GFP-Raji cells compared to the control not undergone co-culture, and particularly the decrease in LMP-1 expression in the shBCL11B-drNK cells was more significant than that in PBMC-NK and NK-92 (FIG. 17C).

Next, the cell-killing ability of the shBCL11B-drNK cells against CEM T cells infected with human immunodeficiency virus (HIV), HEK-293T cells infected with influenza virus, HK2 proximal tubule cells infected with papilloma virus, and SNU449 liver cells infected with hepatitis virus, and the frequency of CD107a⁺ cells expressed through co-culture, were measured in the same manner as above and compared with those of NK-92 cells.

As a result, it was found that the shBCL11B-drNK cells exhibited higher cell-killing ability against all virus-infected cells (FIG. 17D) and a higher frequency of CD107a⁺ cells (FIG. 17E) compared to NK-92 cells, a positive control, at a low E (effector NK cell):T (target cancer cell) ratio as well.

Next, in order to examine the antiviral effect of the shBCL11B-drNK cells against coronavirus, apoptosis was measured in Calu-1 cells (human lung epithelial cells) infected with SARS-CoV-2 and in Calu-1 cells not infected with the virus.

Specifically, 10×10⁴ uninfected Calu-1 cells or SARS-CoV-2 infected Calu-1 cells and 10×10⁴ shBCL11B-drNK cells were dispensed into a 96-well plate and co-cultured for 4 hours in an incubator at 37° C. and 5% CO2. PBMC-NK cells were used as a positive control.

Apoptosis was measured using the APC Annexin V Apoptosis Detection Kit containing propidium iodide (PI) according to the manufacturer's instructions (Biolegend, Cat #: 640932). The co-cultured cells were transferred to an e-tube, washed two times using a cell staining solution and a centrifuge, and suspended in Annexin V binding buffer. Into each tube, 5 μl of APC Annexin V and 10 μl of PI were added, and the reaction was conducted at room temperature for 15 minutes while light was blocked. Annexin V binding buffer was further added, and the measurement was performed using a flow cytometer.

As a result, co-culture with shBCL11B-drNK cells increased apoptosis of the cells infected with SARS-CoV-2 virus compared to the positive control (PMBC-NK cells) (FIG. 18).

Accordingly, it has been found that shBCL11B-drNK cells exhibit excellent antiviral effects.

### Example 9: Antibacterial Efficacy of drNK Cells

In order to examine the antibacterial effect of the shBCL11B-drNK (shRNA #2) cells of Example 1-1 against Gram-negative bacteria, the cell-killing ability against Escherichia coli (E. coli) and the frequency of CD107a⁺ cells expressed through co-culture were measured.

Specifically, one E. coli DH5a colony was taken from an LB agar plate, cultured in LB medium at 37° C. for 16 to 17 hours, then centrifuged at 5000×g for 5 minutes, and then washed with RPMI 1640 medium containing 10% FBS and no antibiotics. The shBCL11B-drNK cells of Example 1-1, PBMC-NK cells as a positive control or NK-92 cells as a positive control were diluted with a culture medium to have densities of 1×10⁴ cells/100 μl, 3×10⁴ cells/100 μl and 9×10⁴ cells/100 μl, and then dispensed into 96-well plates. To the 96-well plates, 3×10⁴ cells/100 μl of E. coli were added, and culture was performed at 37° C. for 0 and 2 hours, respectively. The serially diluted culture solutions were inoculated onto LB agar plates, and cultured at 37° C. for 20 hours, and then the number of E. coli colonies on the LB agar plates was calculated.

The frequency of CD107a⁺ cells was measured 2 hours after the reaction of 3×10⁴ cells/100 μl of shBCL11B-drNK cells with 3×10⁴ cells/100 μl of E. coli (E:T ratio=1:1). The cultured shBCL11B-drNK cells, PBMC-NK cells as a positive control, or NK-92 cells as a positive control were put into FACS buffer containing fluorescent labeled antibodies against CD56 and CD107a and reacted at room temperature for 20 minutes, then the cells were washed and recovered using a centrifuge and subjected to measurement by FACS analysis.

As a result, the number of E. coli colonies was low in both the shBCL11B-drNK cells and the positive controls (PBMC-NK cells and NK-92 cells) (FIG. 19A), and it was found that the cells exhibited high toxicity against E. coli (PBMC-NK: 0 h (0:1); 20,000 pieces on average, 2 h (0:1); 28,000 pieces on average, 2 h (0.3:1); 3,640 pieces on average, 2 h (1:1); 860 pieces on average, and 2 h (3:1); 85 on average, NK-92: 0 h (0:1); 19,825 on average, 2 h (0:1); 28,000 on average, 2 h (0.3:1); 5,200 on average, 2 h (1:1); 3,200 on average, and 2 h (3:1); 88.5 on average, and BCL11B-drNK: 0 h (0:1); 19,825 on average, 2 h (0:1); 28,000 on average, 2 h (0.3:1); 3,540 on average, 2 h (1:1); 397.5 on average, and 2 h (3:1); 80 on average)

It was found that the frequency of CD107a⁺ cells in the shBCL11B-drNK cells was higher than that in the positive controls (PBMC-NK cells and NK-92 cells) at the E:T ratio (1:1) at which the number of E coli colonies was significantly lower than that in the positive controls (FIG. 19B).

Next, in order to examine the antibacterial effect of the shBCL11B-drNK (shRNA #2) cells of Example 1-1 against Gram-positive bacteria, the frequency of CD107a⁺ cells expressed through co-culture with Streptococcus pseudopneumoniae was measured.

Specifically, Streptococcus pseudopneumoniae was suspended in TSB (tryptic soy broth) and washed with RPMI 1640 medium containing 10% FBS and no antibiotics. The shBCL11B-drNK cells of Example 1-1 or NK-92 cells as a positive control were diluted with a culture medium to have a density of 15×10⁴ cells/100 μl and then dispensed into 96-well plates. To the 96-well plates, 15×10⁴ cells/100 μl of Streptococcus pseudopneumoniae were added, culture was performed at 37° C. and 5% CO2 for 2 hours, and then the frequency of CD107a⁺ cells was determined through flow cytometry.

The frequency of CD107a⁺ cells was measured as follows: the shBCL11B-drNK cells or NK-92 cells were put into FACS buffer containing fluorescent labeled antibodies against CD56 and CD107a and reacted at room temperature for 20 minutes, and then the cells were washed and recovered using a centrifuge and subjected to FACS analysis.

As a result, it was found that the test group co-cultured with shBCL11B-drNK cells exhibited a more significant antibacterial effect against Streptococcus pseudopneumoniae compared to NK-92 cells as a positive control (FIG. 19C).

Through this, it has been found that shBCL11B-drNK cells exhibit more significant antibacterial efficacy against Gram-negative and Gram-positive bacteria compared to PBMC-NK cells and NK-92 cells.

### Example 10: Antifungal Efficacy of drNK Cells

In order to examine the antifungal effect of the shBCL11B-drNK (shRNA #2) cells of Example 1-1, the frequency of CD107a⁺ cells expressed through co-culture with Candida albicans was measured.

Specifically, one Candida albicans colony was taken from a YPD agar plate (containing 1% yeast extract, 2% peptone, 2% D-glucose, and 1% agar), and cultured in YPD medium (containing 1% yeast extract, 2% peptone and 2% D-glucose) at 37° C. for 2 hours, then centrifuged at 1000×g for 5 minutes and then washed with RPMI 1640 medium containing 10% FBS and no antibiotics. The shBCL11B-drNK cells of Example 1-1, PBMC-NK cells as a positive control or NK-92 cells as a positive control were diluted with a culture medium to have a density of 2×10⁵ cells/100 μl and then dispensed into 96-well plates. To the 96-well plates, 2×10⁵ cells/100 μl of Candida albicans were added (E:T ratio=1:1), then culture was performed at 37° C. for 6 hours, and then the frequency of CD107a⁺ cells was measured.

The frequency of CD107a⁺ cells was measured as follows: the shBCL11B-drNK cells of Example 1-1, PBMC-NK cells as a positive control or NK-92 cells as a positive control were put into FACS buffer containing fluorescent labeled antibodies against CD56-PE and CD107a-APC and reacted at room temperature for 20 minutes, and then the cells were washed and recovered using a centrifuge and subjected to FACS analysis.

As a result, the frequency of CD107a⁺ cells was highest (20.4%) in a case where shBCL11B-drNK cells and Candida albicans were co-cultured, and the frequency of CD107a⁺ cells was higher in the order of co-culture of PBMC-NK cells and Candida albicans (10.0%), culture of shBCL11B-drNK cells alone (8.5%), co-culture of NK-92 cells and Candida albicans (6.3%), culture of PBMC-NK cells alone (5.6%), and culture of NK-92 cells alone (2.9%) (FIG. 20).

Next, the antifungal effect of the shBCL11B-drNK cells against Aspergillus fumigatus was examined.

Specifically, Aspergillus fumigatus colonies were taken and cultured on a potato dextrose agar (PDA) plate at 25° C. for 5 days, then 12 ml of PBS containing 0.05% Tween 20 was added, and the plate was scraped with flame-sterilized slide glass to obtain a bacterial suspension. The bacterial suspension was filtered through a 40 μm cell strainer, centrifuged at 4000 rpm for 10 minutes, and the supernatant was removed to recover conidia. A process in which the recovered conidia were suspended in PBS and centrifuged and the supernatant was removed was repeated two times for washing, and the conidia were suspended in RPMI 1640 medium. The conidia were inoculated into a 96-well plate and cultured for 4 hours in an incubator at 37° C. and 5% CO2, and co-cultured with the shBCL11B-drNK cells of Example 1-1, PBMC-NK cells as a positive control or NK-92 cells as a positive control at an E:T ratio=1:1 for 6 hours. The supernatant was removed from the 96-well plate, the cells were lysed and washed with sterile distilled water, and then 150 μl of XTT (2,3-bis-(2-methoxy-4-nitro-5-sulfophenyl)-2H-tetrazolium-5-carboxanilide) solution containing coenzyme Q0 (2,3-dimethoxy-5-methyl-p-benzoquinone) was added. Culture was performed for 1 hour in an incubator at 37° C. and 5% CO2, then 100 μl of the supernatant was transferred to a new 96-well plate, and the absorbance was measured at 450 nm and 690 nm using a microplate reader.

As a result, it was found that both the shBCL11B-drNK cells and the positive controls (PBMC-NK cells and NK-92 cells) exhibited antifungal activity, and particularly the shBCL11B-drNK cells exhibited superior antifungal activity (FIG. 21).

From the results of the Examples described above, drNK cells or CAR-drNK cells constructed through the present invention exhibit excellent cell-killing ability against cancer cells or cells infected with viruses, bacteria, and fungi, and can be thus applied as cell therapeutics and compositions for the prevention or treatment of cancer, or infectious diseases caused by bacteria and fungi, and/or inflammatory diseases.

Based on the above description, those skilled in the art to which the present invention pertains will understand that the present invention can be implemented in other specific forms without changing the technical idea or essential features thereof. Therefore, it should be understood that the embodiments described above are not limitative, but illustrative in all respects. The scope of the present invention is defined by the appended claims rather than by the above description, and therefore all changes and modifications that fall within the metes and bounds of the claims or equivalents of such metes and bounds, should be construed as being included in the scope of the present invention.

[Sequence Listing]

| Sequence | Sequence | |
|---|---|---|
| number | name | SEQ |
| 1 | Target | TGAGCCTTCCAGCTACATTTGC (SEQ ID NO: 1) |
| | sense | |
| | sequence of | |
| | shBCL11B# | |
| | 1 | |
| 2 | Target | GTTCTGCGGCAAGACCTTCAAGTTCCAGAG (SEQ ID NO: |
| | sense | 2) |
| | sequence of | |
| | shBCL11B# | |
| | 2 | |
| 3 | Target | GACCTTCAAGTTCCAGAGCAATCTCATCGT (SEQ ID NO: |
| | sense | 3) |
| | sequence of | |
| | shBCL11B# | |
| | 3 | |
| 4 | Target | GTTCAAGAACTGCAGCAACTT (SEQ ID NO: 4) |
| | sense | |
| | sequence of | |
| | shBCL11B# | |
| | 4 | |
| 5 | Target | CAGATCGGCAAGGAGGTGTACCGCTGCGAC (SEQ ID NO: |
| | sense | 5) |
| | sequence of | |
| | shBCL11B# | |
| | 5 | |
| 6 | Target | CCTAACCTGTGTCTGCGAAGTCCTATGGA (SEQ ID NO: 6) |
| | sense | |
| | sequence of | |
| | shBCL11B# | |
| | 6 | |
| 7 | Target | TGGAAACCCGAGGGTTGATTA (SEQ ID NO: 7) |
| | sense | |
| | sequence of | |
| | shBCL11B# | |
| | 7 | |
| 8 | Target | CAGATCGGCAAGGAGGUGUA (SEQ ID NO: 8) |
| | sense | |
| | sequence of | |
| | shBCL11B- | |
| | A | |
| 9 | Target | CCUAACCUGUGUCUGCGAAG (SEQ ID NO: 9) |
| | sense | |
| | sequence of | |
| | shBCL11B- | |
| | B | |
| 10 | Target | GAAACTAGCGGTGTTCTTT (SEQ ID NO: 10) |
| | sense | |
| | sequence of | |
| | shBCL11B- | |
| | C | |
| 11 | Target | CTGAGATTGAGTGTCAGTA (SEQ ID NO: 11) |
| | sense | |
| | sequence of | |
| | shBCL11B- | |
| | D | |
| 12 | sgRNA#1 | GGCAATGTCCCGCCGCAAACAGG (SEQ ID NO: 12) |
| | (forward) | |
| 13 | sgRNA#2 | GCGGGTTGCCCTGTTTGCGGCGG (SEQ ID NO: 13) |
| | (reverse) | |
| 14 | CD8 leader | ATGGCCTTACCAGTGACCGCCTTGCTCCTGCCGCTGGCC |
| | | TTGCTGCTCCACGCCGCCAGGCCG (SEQ ID NO: 14) |
| 15 | CD19 scFv | ATGGCCTTACCAGTGACCGCCTTGCTCCTGCCGCTGGCC |
| | | TTGCTGCTCCACGCCGCCAGGCCGGACATCCAGATGAC |
| | | ACAGACTACATCCTCCCTGTCTGCCTCTCTGGGAGACAG |
| | | AGTCACCATCAGTTGCAGGGCAAGTCAGGACATTAGTAA |
| | | ATATTTAAATTGGTATCAGCAGAAACCAGATGGAACTGTTA |
| | | AACTCCTGATCTACCATACATCAAGATTACACTCAGGAGT |
| | | CCCATCAAGGTTCAGTGGCAGTGGGTCTGGAACAGATTA |
| | | TTCTCTCACCATTAGCAACCTGGAGCAAGAAGATATTGCC |
| | | ACTTACTTTTGCCAACAGGGTAATACGCTTCCGTACACGT |
| | | TCGGAGGGGGGACCAAGCTGGAGATCACAGGTGGCGGT |
| | | GGCTCGGGCGGTGGTGGGTCGGGTGGCGGCGGATCTG |
| | | AGGTGAAACTGCAGGAGTCAGGACCTGGCCTGGTGGCG |
| | | CCCTCACAGAGCCTGTCCGTCACATGCACTGTCTCAGGG |
| | | GTCTCATTACCCGACTATGGTGTAAGCTGGATTCGCCAGC |
| | | CTCCACGAAAGGGTCTGGAGTGGCTGGGAGTAATATGGG |
| | | GTAGTGAAACCACATACTATAATTCAGCTCTCAAATCCAGA |
| | | CTGACCATCATCAAGGACAACTCCAAGAGCCAAGTTTTCT |
| | | TAAAAATGAACAGTCTGCAAACTGATGACACAGCCATTTA |
| | | CTACTGTGCCAAACATTATTACTACGGTGGTAGCTATGCTA |
| | | TGGACTACTGGGGCCAAGGAACCTCAGTCACCGTCTCCT |
| | | CA (SEQ ID NO: 15) |
| 16 | MSLN scFv | CAAGTCCAACTCGTTCAATCAGGCGCAGAAGTCGAAAAG |
| | | CCCGGAGCATCAGTCAAAGTCTCTTGCAAGGCTTCCGGC |
| | | TACACCTTCACGGACTACTACATGCACTGGGTGCGCCAG |
| | | GCTCCAGGCCAGGGACTGGAGTGGATGGGATGGATCAA |
| | | CCCGAATTCCGGGGGAACTAACTACGCCCAGAAGTTTCA |
| | | GGGCCGGGTGACTATGACTCGCGATACCTCGATCTCGAC |
| | | TGCGTACATGGAGCTCAGCCGCCTCCGGTCGGACGATAC |
| | | CGCCGTGTACTATTGTGCGTCGGGATGGGACTTCGACTA |
| | | CTGGGGGCAGGGCACTCTGGTCACTGTGTCAAGCGGAG |
| | | GAGGTGGATCAGGTGGAGGTGGAAGCGGGGGAGGAGG |
| | | TTCCGGCGGCGGAGGATCAGATATCGTGATGACGCAATC |
| | | GCCTTCCTCGTTGTCCGCATCCGTGGGAGACAGGGTGA |
| | | CCATTACTTGCAGAGCGTCCCAGTCCATTCGGTACTACCT |
| | | GTCGTGGTACCAGCAGAAGCCGGGGAAAGCCCCAAAAC |
| | | TGCTTATCTATACTGCCTCGATCCTCCAAAACGGCGTGCC |
| | | ATCAAGATTCAGCGGTTCGGGCAGCGGGACCGACTTTAC |
| | | CCTGACTATCAGCAGCCTGCAGCCGGAAGATTTCGCCAC |
| | | GTACTACTGCCTGCAAACCTACACCACCCCGGACTTCGG |
| | | ACCTGGAACCAAGGTGGAGATCAAG (SEQ ID NO: 16) |
| 17 | HER2 scFv | CAGGTGCAGCTGCAGCAGAGCGGCCCTGAGCTGAAGAA |
| | | GCCCGGCGAGACAGTCAAGATCAGCTGCAAGGCCAGCG |
| | | GCTACCCCTTCACCAACTACGGCATGAACTGGGTGAAAC |
| | | AGGCCCCAGGCCAGGGACTGAAGTGGATGGGCTGGATC |
| | | AACACCAGCACCGGCGAGAGCACCTTCGCCGACGACTT |
| | | CAAGGGCAGATTCGACTTCAGCCTGGAAACCAGCGCCAA |
| | | CACCGCCTACCTGCAGATCAACAACCTGAAGAGCGAGGA |
| | | CAGCGCCACCTACTTTTGCGCCAGATGGGAGGTG |
| | | TACCACGGCTACGTGCCCTACTGGGGCCAGGGCACCAC |
| | | CGTGACCGTGTCCAGCGGCGGAGGGGGCTCTGGCGGC |
| | | GGAGGATCTGGGGGAGGGGGCAGCGACATCCAGCTGAC |
| | | CCAGAGCCACAAGTTTCTGAGCACCAGCGTGGGCGACC |
| | | GGGTGTCCATCACCTGCAAAGCCAGCCAGGACGTGTACA |
| | | ACGCCGTGGCCTGGTATCAGCAGAAGCCTGGCCAGAGC |
| | | CCCAAGCTGCTGATCTACAGCGCCAGCAGCCGGTACACC |
| | | GGCGTGCCCAGCAGGTTCACCGGCAGCGGCAGCGGCC |
| | | CAGACTTCACCTTCACCATCAGCAGCGTGCAGGCCGAG |
| | | GACCTGGCCGTGTACTTCTGCCAGCAGCACTTCCGGACC |
| | | CCCTTCACCTTCGGCTCCGGCACCAAGCTGGAAATCAAG |
| | | (SEQ ID NO: 17) |
| 18 | CD8 hinge | ACCACGACGCCAGCGCCGCGACCACCAACACCGGCGCC |
| | | CACCATCGCGTCGCAGCCCCTGTCCCTGCGCCCAGAGG |
| | | CGTGCCGGCCAGCGGCGGGGGGCGCAGTGCACACGAG |
| | | GGGGCTGGACTTCGCCTGTGAT (SEQ ID NO: 18) |
| 19 | CD8 | ATCTACATCTGGGCGCCCTTGGCCGGGACTTGTGGGGTC |
| | transmembrane | CTTCTCCTGTCACTGGTTATCACCCTTTACTGC (SEQ ID |
| | domain | NO: 19) |
| 20 | Fc gamma | AGACGACTCAAGATCCAGGTCCGAAAGGCAGCTATAGCC |
| | receptor | AGCCGTGAGAAAGCAGATGCTGTCTACACGGGCCTGAAC |
| | | ACCCGGAGCCAGGAGACATATGAGACTCTGAAGCATGAG |
| | | AAACCACCCCAGGGATCCGGAAGT (SEQ ID NO: 20) |
| 21 | CD28 | AGGAGTAAGAGGAGCAGGCTCCTGCACAGTGACTACATG |
| | intracellular | AACATGACTCCCCGCCGCCCCGGGCCCACCCGCAAGCA |
| | domain | TTACCAGCCCTATGCCCCACCACGCGACTTCGCAGCCTA |
| | | TCGCTCC (SEQ ID NO: 21) |
| 22 | CD3 zetta | AGAGTGAAGTTCAGCAGGAGCGCAGACGCCCCCGCGTA |
| | | CCAGCAGGGCCAGAACCAGCTCTATAACGAGCTCAATCT |
| | | AGGACGAAGAGAGGAGTACGATGTTTTGGACAAGAGACG |
| | | TGGCCGGGACCCTGAGATGGGGGGAAAGCCGAGAAGG |
| | | AAGAACCCTCAGGAAGGCCTGTACAATGAACTGCAGAAA |
| | | GATAAGATGGCGGAGGCCTACAGTGAGATTGGGATGAAA |
| | | GGCGAGCGCCGGAGGGGCAAGGGGCACGATGGCCTTT |
| | | ACCAGGGTCTCAGTACAGCCACCAAGGACACCTACGACG |
| | | CCCTTCACATGCAGGCCCTGCCCCCTCGC (SEQ ID NO: |
| | | 22 |
| 23 | IRES | GCCCCTCTCCCTCCCCCCCCCCTAACGTTACTGGCCGAA |
| | | GCCGCTTGGAATAAGGCCGGTGTGCGTTTGTCTATATGTT |
| | | ATTTTCCACCATATTGCCGTCTTTTGGCAATGTGAGGGCC |
| | | CGGAAACCTGGCCCTGTCTTCTTGACGAGCATTCCTAGG |
| | | GGTCTTTCCCCTCTCGCCAAAGGAATGCAAGGTCTGTTG |
| | | AATGTCGTGAAGGAAGCAGTTCCTCTGGAAGCTTCTTGA |
| | | AGACAAACAACGTCTGTAGCGACCCTTTGCAGGCAGCGG |
| | | AACCCCCCACCTGGCGACAGGTGCCTCTGCGGCCAAAA |
| | | GCCACGTGTATAAGATACACCTGCAAAGGCGGCACAACC |
| | | CCAGTGCCACGTTGTGAGTTGGATAGTTGTGGAAAGAGT |
| | | CAAATGGCTCACCTCAAGCGTATTCAACAAGGGGCTGAA |
| | | GGATGCCCAGAAGGTACCCCATTGTATGGGATCTGATCTG |
| | | GGGCCTCGGTGCACATGCTTTACATGTGTTTAGTCGAGG |
| | | TTAAAAAACGTCTAGGCCCCCCGAACCACGGGGACGTG |
| | | GTTTTCCTTTGAAAAACACGATGATAAT (SEQ ID NO: 23) |
| 24 | GPF | ATGGTGAGCAAGGGCGAGGAGCTGTTCACCGGGGTGGT |
| | | GCCCATCCTGGTCGAGCTGGACGGCGACGTAAACGGCC |
| | | ACAAGTTCAGCGTGTCCGGCGAGGGCGAGGGCGATGCC |
| | | ACCTACGGCAAGCTGACCCTGAAGTTCATCTGCACCACC |
| | | GGCAAGCTGCCCGTGCCCTGGCCCACCCTCGTGACCAC |
| | | CCTGACCTACGGCGTGCAGTGCTTCAGCCGCTACCCCG |
| | | ACCACATGAAGCAGCACGACTTCTTCAAGTCCGCCATGC |
| | | CCGAAGGCTACGTCCAGGAGCGCACCATCTTCTTCAAGG |
| | | ACGACGGCAACTACAAGACCCGCGCCGAGGTGAAGTTC |
| | | GAGGGCGACACCCTGGTGAACCGCATCGAGCTGAAGGG |
| | | CATCGACTTCAAGGAGGACGGCAACATCCTGGGGCACAA |
| | | GCTGGAGTACAACTACAACAGCCACAACGTCTATATCATG |
| | | GCCGACAAGCAGAAGAACGGCATCAAGGTGAACTTCAAG |
| | | ATCCGCCACAACATCGAGGACGGCAGCGTGCAGCTCGC |
| | | CGACCACTACCAGCAGAACACCCCCATCGGCGACGGCC |
| | | CCGTGCTGCTGCCCGACAACCACTACCTGAGCACCCAGT |
| | | CCGCCCTGAGCAAAGACCCCAACGAGAAGCGCGATCAC |
| | | ATGGTCCTGCTGGAGTTCGTGACCGCCGCCGGGATCAC |
| | | TCTCGGCATGGACGAGCTGTACAAG (SEQ ID NO: 24) |
| 25 | RHA | AAAGGCGGCTGCGGAATCCTAGGACTGGCGCGGCCGGC |
| | | ACCCTGCTGGGCCGGGCGCAGGGACCGGGGACCCGGA |
| | | GCCGGCGGCCGCCCCTGGCCCTACGGCTCCCCCAGCC |
| | | GGAACGCACCCCGCTCTCCTCCGCCCGCGCCGTCAGCG |
| | | CGGACCCACGCGCTCGCCAACTTTTCCCCACTTCCCGG |
| | | CTCCCCCTCCCCCTCCGCTCCCCCAGCCCCCTCCCCCT |
| | | CCTCTCCAAACCCCGCCACCAGCGCCGCCGCCGCCACA |
| | | CACGCCGCTCGGAGGGGCGAGCGTCCAGCCGGGCTCG |
| | | GCGCGCACACACACACACTCCTCCAGCCTGCATGCCCC |
| | | CTCCCCGGCCCGGAGCCGGCTCCGCAGGCCCCCGAGC |
| | | CCGAGGCGCGTCCGGCTGCTCGGCGCCCCAACTCCCC |
| | | GGGCTGCAAAGAAACTTTCCTATGGCCCCCGCCCCCCG |
| | | CCATGCTCCAGGCCGACGCCGTAGACTCTGCCAGCCAG |
| | | CGGGCGGCCCCGGCGCCTGGCCAGGCTCGGCTGTTCC |
| | | GGGCTCGGTGTCCCCAGCCCCAGACGCCCGGAGCCCC |
| | | ATCTCCGGCCCCTCGCGCGCACTCCGCAGACACTTAC |
| | | (SEQ ID NO: 25) |
| 26 | LHA | TGCCCCGGCATCTATTCTGGCATCGCCCGGAGAGCTGCA |
| | | CTGATGGGGGGAGCCGGGGGAGGGGGTCCGAGCCGCC |
| | | GCCGCGCCGCTGCCGCCGCTGCCGCCGCCGCCGCCGC |
| | | CGCCGCACCTCCTCCTCTGCCCGGGTTGGTGTTTTTTTT |
| | | CCCTTCCTCTCTTTCCCTCTCTTCCTCCTCTTCTTCTTCTT |
| | | TATTTTGCTCTTTCTTCTATGCTGTTTTTTGTTTTGTTTGCA |
| | | AAAAGAAAAAAAAGGGAAGAAAAGCAAGAAAAACCTCTC |
| | | GATCTAAAATAAGAAAAAGAGGCAAAAAAAAAAAAAACTG |
| | | CTGTTGCTTTCCGCGGACTGGCTGGTTTCTTTAAAAATAT |
| | | ATTCTTTCGAAGGAAAAAAAATCTCTTACACTTCTTCAAAC |
| | | TGCTTGGCCTCTTGCACTTGCAAATGTCTTCTTGAACTTA |
| | | AACTGGGTTTTGCACCGGCTCCTGACACTTTCTTTCAGG |
| | | GTCTGGTAGGTGGAAAGCGCACTTCTACCAGGAGGGGA |
| | | AAAAAAATGCAAACAAATAAAAAAATAAAAGAAGAAAAAGC |
| | | AAAGGAAAAAAAAAAGCAAAGAAAACTTGGGGACTTGTC |
| | | TCGTACCC (SEQ ID NO: 26) |
| 27 | SFFV | GTAACGCCATTTTGCAAGGCATGGAAAAATACCAAACCAA |
| | promoter | GAATAGAGAAGTTCAGATCAAGGGCGGGTACATGAAAATA |
| | | GCTAACGTTGGGCCAAACAGGATATCTGCGGTGAGCAGT |
| | | TTCGGCCCCGGCCCGGGGCCAAGAACAGATGGTCACCG |
| | | CAGTTTCGGCCCCGGCCCGAGGCCAAGAACAGATGGTC |
| | | CCCAGATATGGCCCAACCCTCAGCAGTTTCTTAAGACCCA |
| | | TCAGATGTTTCCAGGCTCCCCCAAGGACCTGAAATGACC |
| | | CTGCGCCTTATTTGAATTAACCAATCAGCCTGCTTCTCGC |
| | | TTCTGTTCGCGCGCTTCTGCTTCCCGAGCTCTATAAAAGA |
| | | GCTCACAACCCCTCACTCGGCGCGCCAGTCCTCCGACA |
| | | GACTGAGTCGCCCGGG (SEQ ID NO: 27) |
| 28 | Poly A | GCCCCTCTCCCTCCCCCCCCCCTAACGTTACTGGCCGAA |
| | signal | GCCGCTTGGAATAAGGCCGGTGTGCGTTTGTCTATATGTT |
| | | ATTTTCCACCATATTGCCGTCTTTTGGCAATGTGAGGGCC |
| | | CGGAAACCTGGCCCTGTCTTCTTGACGAGCATTCCTAGG |
| | | GGTCTTTCCCCTCTCGCCAAAGGAATGCAAGGTCTGTTG |
| | | AATGTCGTGAAGGAAGCAGTTCCTCTGGAAGCTTCTTGA |
| | | AGACAAACAACGTCTGTAGCGACCCTTTGCAGGCAGCGG |
| | | AACCCCCCACCTGGCGACAGGTGCCTCTGCGGCCAAAA |
| | | GCCACGTGTATAAGATACACCTGCAAAGGCGGCACAACC |
| | | CCAGTGCCACGTTGTGAGTTGGATAGTTGTGGAAAGAGT |
| | | CAAATGGCTCACCTCAAGCGTATTCAACAAGGGGCTGAA |
| | | GGATGCCCAGAAGGTACCCCATTGTATGGGATCTGATCTG |
| | | GGGCCTCGGTGCACATGCTTTACATGTGTTTAGTCGAGG |
| | | TTAAAAAACGTCTAGGCCCCCCGAACCACGGGGACGTG |
| | | GTTTTCCTTTGAAAAACACGATGATAAT (SEQ ID NO: 28) |
| 29 | LHA primer | ACC GAA CCG GGG CAG TTT TA (SEQ ID NO: 29) |
| 30 | SFFV | TTT TCA TGT ACC CGC CCT TGAT (SEQ ID NO: 30) |
| | promoter | |
| | primer | |
| 31 | LMP-1 | TCCTCCTGTTTCTGGCGATT (SEQ ID NO: 31) |
| | forward | |
| 32 | LMP-1 | GGAGTCATCGTGGTGGTGTTC (SEQ ID NO: 32) |
| | reverse | |
| 33 | GFP | ATGGTGAGCAAGGGCGAGGAG (SEQ ID NO: 33) |
| | forward | |
| 34 | GFP | CGGTGGTGCAGATGAACTTCAGG (SEQ ID NO: 34) |
| | reverse | |

### Reference to a “Sequence Listing” Submitted as an XML File

The material in the XML file, named “HANOL-70043-Sequence-Listing.xml”, created Mar. 7, 2024, file size of 57,344 bytes, is hereby incorporated by reference.

1. A method for preparing a directly reprogrammed natural killer (drNK) cell, comprising:
a) inhibiting BCL11B gene expression in an isolated cell; and
b) culturing the cell in step a) in a medium containing a cytokine and a growth factor to convert the cell into an NK (natural killer) cell.

2. The method according to claim 1, wherein step a) is a step of inhibiting BCL11B gene expression in an isolated cell by introducing any one or more selected from the following i) to iii) into the cell:
i) shRNA (short hairpin RNA),
ii) siRNA (short interfering RNA), and
iii) CRISPR (clustered regularly interspaced short palindromic repeats)/Cas system.

3. The method according to claim 1, wherein the isolated cell in step a) is a somatic cell excluding an NK cell.

4. The method according to claim 2, wherein step a) is a step of inhibiting BCL11B gene expression in an isolated cell by introducing any one or more selected from the following i) to iii) into the cell:
i) a target sense sequence of the shRNA is any one or more selected from the group consisting of SEQ ID NOs: 1 to 7,
ii) a target sense sequence of the siRNA is any one or more selected from the group consisting of SEQ ID NOs: 8 to 11, and
iii) the gRNA is any one or more selected from the group consisting of SEQ ID NOs: 12 and 13.

5. The method according to claim 2, wherein the CRISPR/Cas system is CRISPR/Cas9-gRNA-BCL11B, and wherein the CRISPR/Cas9-gRNA-BCL11B is introduced in any one or more steps selected from step a) and step b).

6. The method according to claim 1, wherein the growth factor in b) is any one or more selected from the group consisting of EGF (epidermal growth factor), PDGF-AA (platelet-derived growth factor-AA), IGF-1 (insulin-like growth factor 1), TGF-β (transforming growth factor-3), FGF (fibroblast growth factor), SCF (stem cell factor), and FLT3L (FMS-like tyrosine kinase ligand).

7. The method according to claim 1, wherein the cytokine in b) is any one or more selected from the group consisting of IL (interleukin)-2, IL-3, IL-5, IL-6, IL-7, IL-11, IL-15, IL-21, BMP4 (bone morphogenetic protein 4), activin A, notch ligand, G-CSF (granulocyte-colony stimulating factor), and SDF-1 (stromal cell-derived factor-1).

8. The method according to claim 1, wherein the medium in b) further contains any one or more selected from the group consisting of a GSK3β (glycogen synthase kinase 3β) inhibitor, a PDK1 (3-phosphoinositide-dependent kinase 1) inhibitor, and an AHR (aryl hydrocarbon receptor) inhibitor.

9. A drNK cell prepared by the method according to claim 1.

10. The cell according to claim 9, expressing any one or more selected from the group consisting of CD56⁺, CD3⁻, and a combination thereof.

11. A method for preparing a CAR-drNK cell, comprising additionally introducing a CAR gene in any one or more steps selected from a) and b) in the method of claim 1.

12. The method according to claim 11, wherein the CAR gene is any one or more selected from the group consisting of CD19-CAR, MSLN-CAR, and HER2-CAR.

13. The method according to claim 11, wherein the CAR gene is introduced into a BCL11B knock-out base sequence through knock-in.

14. The method according to claim 11, wherein the CAR gene is any one or more selected from the group consisting of:
i) a CAR gene containing CD8 leader, CD19 scFv, CD8 hinge, CD8 transmembrane domain and Fc-γ (gamma) receptor;
ii) a CAR gene containing CD8 leader, MSLN (mesothelin) scFv, CD8 hinge, CD8 transmembrane domain, CD28 intracellular domain, CD3ζ and IRES; and
iii) a CAR gene containing CD8 leader, HER2 (human epidermal growth factor receptor 2) scFv, CD8 hinge, CD8 transmembrane domain, CD28 intracellular domain, CD3ζ and IRES.

15. A CAR-drNK cell prepared by the method according to claim 11.

16. The cell according to claim 15, expressing any one or more selected from the group consisting of CD56⁺, CD3⁻, and a combination thereof.

17. A composition comprising a cell prepared by the method according to claim 1 and/or a cell prepared by additionally introducing a CAR gene into the cell as an active ingredient.

18. The composition according to claim 17, wherein the composition is any one or more selected from the group consisting of:
i) a cell therapeutic composition for prevention or treatment of cancer;
ii) a pharmaceutical composition for prevention or treatment of cancer;
iii) a cell therapeutic composition for prevention or treatment of an infectious disease and/or an inflammatory disease; and
iv) a pharmaceutical composition for prevention or treatment of an infectious disease and/or an inflammatory disease.

19. A method for treating cancer, comprising administering a composition containing a cell prepared by the method according to claim 1 and/or a cell prepared by additionally introducing a CAR gene into the cell as an active ingredient to a subject in need thereof.

20. A method for treating an infectious disease and/or an inflammatory disease, comprising administering a composition containing a cell prepared by the method according to claim 1 and/or a cell prepared by additionally introducing a CAR gene into the cell as an active ingredient to a subject in need thereof.
